(19) United States
(12) Patent Application Publication
(10) Pub. No.: US 2009/0131498 A1
(43) Pub. Date: May 21, 2009

(54) Enantioselective Synthesis of Merrilactone and Its Analogs

(76) Inventors: Samuel J. Danishefsky, Englewood, NJ (US); Zhaoyang Meng, New York, NY (US); Vladimir Birman, Brentwood, MO (US)

Correspondence Address: COOPER & DUNHAM, LLP, 30 Rockefeller Plaza, 20th Floor, NEW YORK, NY, 10112, US

(21) Appl. No.: 11/795,705

(22) PCT Filed: Jan. 18, 2006
(86) PCT No.: PCT/US2006/002643

§ 371 Date: Aug. 11, 2008

Related U.S. Application Data
(60) Provisional application No. 60/645,501, filed on Jan. 18, 2005.

(51) Int. Cl. A61K 31/365; C07D 493/02; C07D 491/02; C07C 69/757; C07C 49/573; A61P 25/00; C07D 303/02; C07D 307/93; C07D 409/02; A61K 31/407

(52) U.S. Cl. 514/410; 549/298; 549/297; 548/421; 560/120; 568/379; 549/545; 549/459; 549/465; 549/22; 514/468

(57) ABSTRACT

This invention provides a method of synthesizing enantioenriched merrilactone A and enantiopure merrilactone A, as well as an improved method of synthesizing racemic merrilactone. This invention also provides intermediate compounds and methods of treating peripheral neuropathies

This application claims the benefit of U.S. Provisional Application No. 60/645,501, filed Jan. 18, 2005, the contents of which are incorporated hereby by reference into the subject application.

The invention disclosed herein was made with Government support under grant no. HL 25848 from the National Institutes of Health. Accordingly, the U.S. Government has certain rights in this invention.

Throughout this application, various publications are referenced by numbers in parentheses, and their full citations may be found at the end of the specification. The disclosures of these publications in their entireties are hereby incorporated by reference into this application in order to more fully describe the state of the art as known to those skilled therein as of the date of the invention described and claimed herein.

### BACKGROUND OF THE INVENTION

In 2002, the total synthesis of (±)-merrilactone A (or racemic merrilactone A) (1) was reported. Also, see US 2004-0006121 A1, which is hereby incorporated by reference. Merrilactone-A has propeller-like topology, consisting of five interlocking cis fusions (including two γ-lactones and an oxetane). Six stereogenic bridgehead centers serve as the anchor points of these fusions.

Merrilactone A, the naturally occurring enantiomeric form of which is referred to herein as (+)-Merrilactone A, is a member of a class of nonpeptidal neurotrophic factors.

Maintenance of appropriate levels of polypeptidal neurotrophic factors in the central nervous system can be critical in promoting neuronal cell viability. Administration of polypeptidal neurotrophic factors to damaged neuronal cells can lead, in vitro, to substantially restored phenotypes (2). Unfortunately, however, the natural polypeptidal neurotrophic factors have performed poorly in in vivo settings (2). These failures have been ascribed to the usual transport and pharmacostability issues that beset the use of polypeptides. Thus, our laboratory has been studying the synthesis of potential nonpeptidal, small molecules with neurotrophic activity. Such compounds should overcome some of the pharmacostability issues that plague polypeptidal neurotrophic factors. Fukuyama et al. described promising activity for merrilactone A in a neurite growth assay (3). It was these considerations that prompted our first experiments directed towards the total synthesis of merrilactone A, which was indeed accomplished (see FIG. 1) (1).

There were several areas where significant improvement in the earlier synthesis would be helpful. Thus, while compound 2 of FIG. 1 could be synthesized in reasonable yields through Diels-Alder cycloaddition, its conversion to γ-lactone 3 was not straightforward. Various attempted ring openings of the anhydride were non-regioselective. In the event, the isomeric products arising from both modes of ring opening (see arrows, FIG. 1) could be individually converted to the desired 3.

A second difficulty arose at the level of relative stereochemistry. The transformation of 4 to 5b (FIG. 1) by Claisen rearrangement was never realized in a selective fashion, despite many attempts. At best, we could obtain only a 1.8:1 ratio of isomers (5b:5a, FIG. 1) in the desired sense. Moreover, the synthesis produced racemic merrilactone A. In the context of launching a SAR study in this family of compounds, it would certainly be of interest to be able to evaluate merrilactone A in its enantiomerically pure form.

This invention provides a method of synthesizing enantioenriched merrilactone A and enatiopure merrilactone A, as well as an improved method of synthesizing racemic merrilactone A.

### SUMMARY

In one embodiment, this invention provides an enantioenriched composition comprising a compound having the structure:

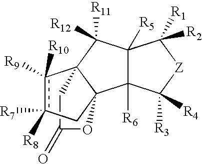

wherein Z is O or >N—X, where X is H, straight or branched substituted or unsubstituted alkyl, alkenyl or alkynyl, or acyl, carbamoyl, cycloalkyl, aryl, heterocycloalkyl, heteroaryl, aralkyl, amino, alkyl amino, or dialkyl amino;
wherein each of R₁ and R₂ is H or R₁ and R₂ together are ═O;
wherein each of R₃ and R₄ is H or R₃ and R₄ together are ═O;
wherein each of R₅ and R₆ are, independently, H, alkyl, aralkyl, or aryl;
wherein each of R₇ and R₈ is, independently, H, OH, or OR₁₄, where R₁₄ is alkyl or —C(O)—R₁₅,
where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino, wherein each R₁₆ is straight or branched, substituted or unsubstituted alkyl, alkenyl or alkynyl, cycloalkyl, aryl, heterocycloalkyl, heteroaryl, aralkyl, or amino; and wherein R₁₇ is straight or branched, unsubstituted alkyl, alkenyl or alkynyl, cycloalkyl, aryl, heterocycloalkyl, heteroaryl, aralkyl, or amino,
or wherein R₇ and R₉ together with the carbons to which each is attached form an oxirane moiety; wherein each of R₉ and R₁₀ is, independently, H, alkyl, OH, or OR₁₃, where R₁₃ is an alkyl, an acyl, or an amide, or R₉ and R₁₀ together are ═CH₂, or wherein R₈ and R₁₀ together with the carbons to which each is attached form an oxirane moiety; wherein if one of R₇ or R₈ and one of R₉ or R₁₀ is absent, a double bond is formed as indicated by the broken line; and wherein each of R₁₁ and R₁₂ is, independently, H, OH, or OR₁₃, where R₁₃ is an alkyl, an acyl, or an amide, or R₁₁ and R₁₂ together are ═O, or wherein R₁₂ and R₁₀ together with the carbons to which each is attached form an oxetane moiety, or an enantiomer, tautomer or salt of the compound.

### BRIEF DESCRIPTION OF THE FIGURES

FIG. 1: This figure shows the Birman-Danishefsky synthesis of racemic merrilactone A.

FIG. 2: This figure shows the synthesis of the key meso intermediate 14. (a) 180° C., neat; then MeOH, reflux; PhH/MeOH; TMSCHN₂, 92% for one-pot reaction; (b) LDA, HMPA, MeI, THF, −78° C. →rt, 95%; (c) LAH, THF, reflux, (d) Na, NH₃, THF/EtOH, −78° C.; acidic work-up, 72% for 2 steps; (e) 2,2-dimethoxypropane, acetone, pTsOH; (f) NaH, (EtO)₂POCH₂CO₂Et, THF, 86% for 2 steps; (g) Mg, MeOH; acidic work-up, 77%.

FIG. 3: This figure shows the Baeyer-Villiger Oxidation of Compound 17. (a) mCPBA, CH₂Cl₂, 90%; (b) PDC, DMF; (c) K₂CO₃, MeI, acetone, reflux, 70% for 2 steps; (d) MMPP, MeOH, OC→rt, 88%; (e) DCC, mCPBA, 0° C.→rt, 83%; (f) PhH, reflux; (g) K₂CO₃, MeOH, 70%.

FIG. 4: This figure shows the completion of the synthesis of racemic intermediate. (a) BF₃.OEt₂, HS(CH₂)₃SH, CH₂Cl₂, 50%; (b) PhI(OCF₃CO₂)₂, CH₃CN—H₂O, 50%; (c) NaBH₄, MeOH, 0° C.; (d) o-NO₂C₆H₄SeCN, Bu₃P, THF, then 30% H₂O₂, 86%; (e) TBSOTf, Et₃N, CH₂Cl₂, 76%; (f) LiOH, MeOH/H₂O, then 12, sat. NaHCO₃/THF, 75%.

FIG. 5: This figure shows desymmetrization of meso Compound 14. (a) DMDO, CH₂Cl₂, 0.5 to 1 hr; (b) (S,S)-[Co^{III}(salen)]-OAc, −78° C. for 2 days, then −25° C. for 2 days, THF.

FIG. 6: This figure shows a generalized route of synthesizing racemic merrilactone A.

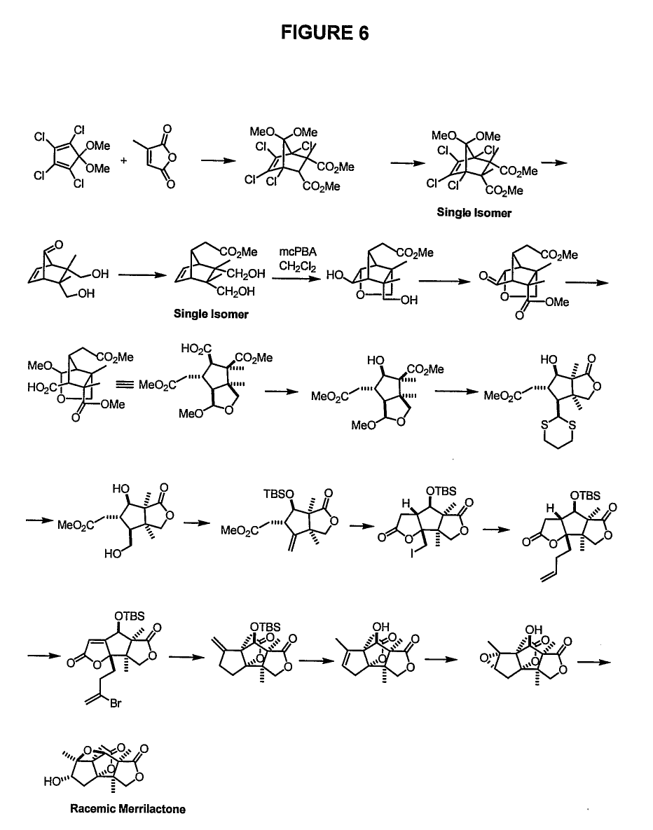

FIG. 7: This figure shows a generalized route of synthesizing enantiomeric merrilactone A. Either enantiomer can be produced by choosing the appropriate R,R or S,S catalyst.

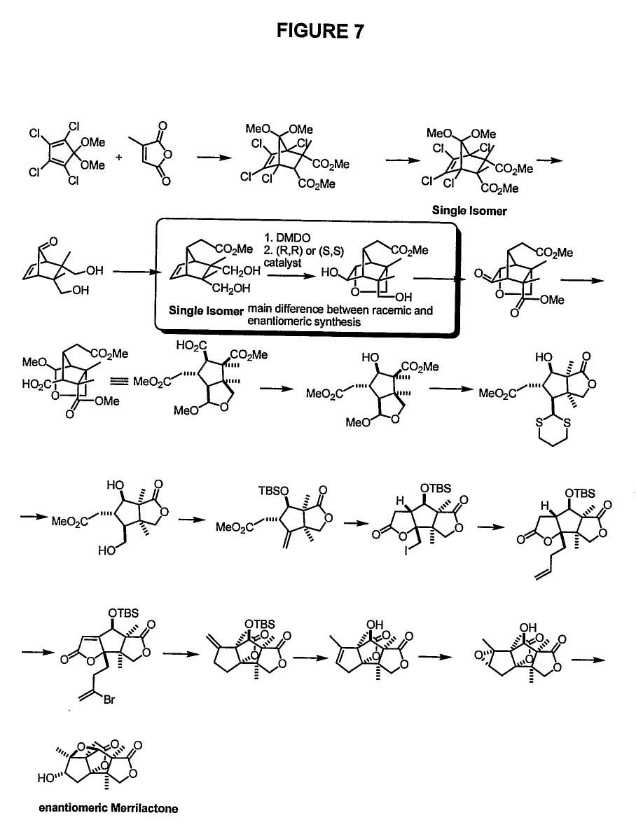

FIG. 8: This figure shows a generalized route of synthesizing certain merrilactone A analogs. In this synthesis R₅ and R₆ may be the same group, as shown in the figure wherein “R₅” has been used for both positions, or may be different independent groups.

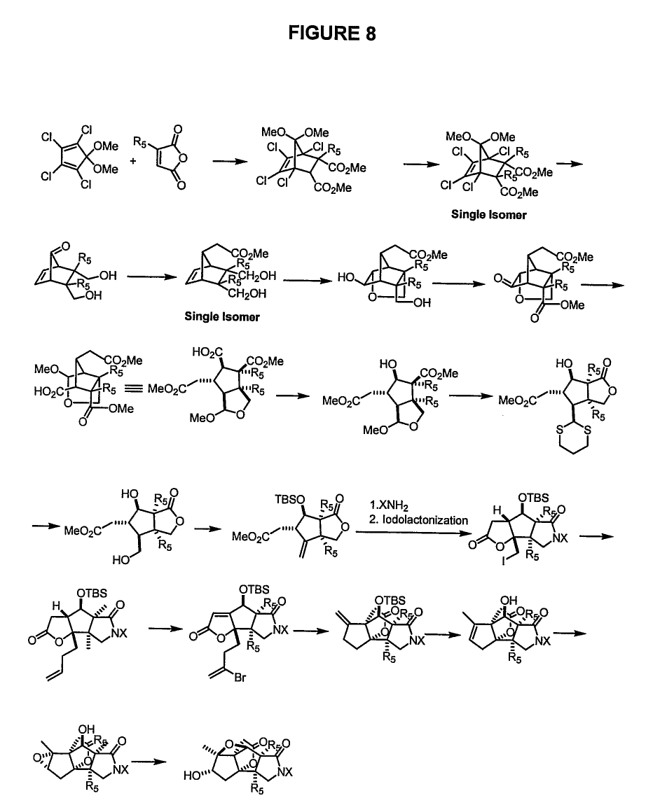

FIG. 9: This figure shows a generalized route of synthesizing certain merrilactone A analogs. In this synthesis R₅ and R₆ may be the same group, as shown in the figure wherein “R₅” has been used for both positions, or may be different independent groups.

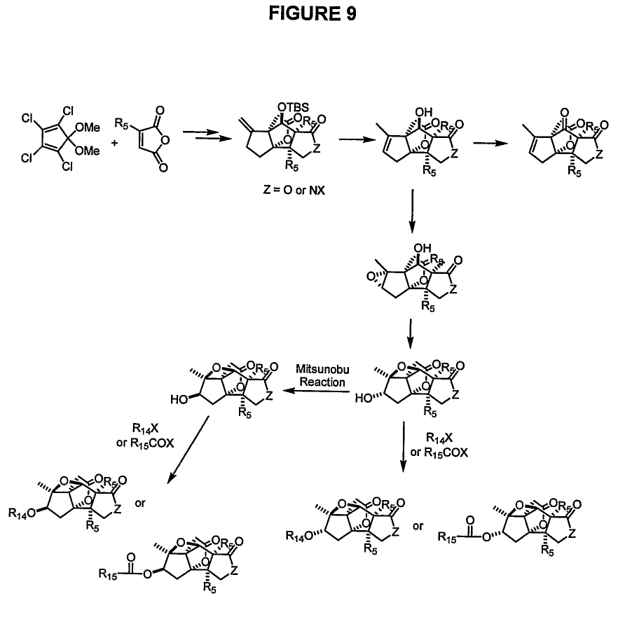

### DETAILED DESCRIPTION

This invention provides an enantioenriched composition comprising a compound having the structure:

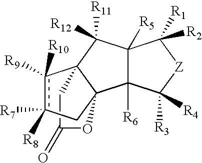

wherein Z is O or >N—X, where X is H, straight or branched substituted or unsubstituted alkyl, alkenyl or alkynyl, or acyl, carbamoyl, cycloalkyl, aryl, heterocycloalkyl, heteroaryl, aralkyl, amino, alkyl amino, or dialkyl amino;
wherein each of R₁ and R₂ is H or R₁ and R₂ together are ═O;
wherein each of R₃ and R₄ is H or R₃ and R₄ together are ═O;
wherein each of R₅ and R₆ are, independently, H, alkyl, aralkyl, or aryl;
wherein each of R₇ and R₈ is, independently, H, OH, or OR₁₄, where R₁₄ is alkyl or —C(O)—R₁₅,
where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino, wherein each R₁₆ is straight or branched, substituted or unsubstituted alkyl, alkenyl or alkynyl, cycloalkyl, aryl, heterocycloalkyl, heteroaryl, aralkyl, or amino; and wherein R₁₇ is straight or branched, unsubstituted alkyl, alkenyl or alkynyl, cycloalkyl, aryl, heterocycloalkyl, heteroaryl, aralkyl, or amino,
or wherein R₇ and R₉ together with the carbons to which each is attached form an oxirane moiety; wherein each of R₉ and R₁₀ is, independently, H, alkyl, OH, or OR₁₃, where R₁₃ is an alkyl, an acyl, or an amide, or R₉ and R₁₀ together are ═CH₂, or wherein R⁸ and R₁₀ together with the carbons to which each is attached form an oxirane moiety; wherein if one of R₇ or R₈ and one of R₉ or R₁₀ is absent, a double bond is formed as indicated by the broken line; and wherein each of R₁₁ and R₁₂ is, independently, H, OH, or OR₁₃, where R₁₃ is an alkyl, an acyl, or an amide, or R₁₁ and R₁₂ together are ═O, or wherein R₁₂ and R₁₀ together with the carbons to which each is attached form an oxetane moiety, or an enantiomer, tautomer or salt of the compound.

This invention further provides the instant composition, wherein in the compound when X is a substituted alkyl, substituents are selected from OH, oxo, halogen, alkoxy, diaklyamino or heterocyclyl; wherein in the compound Z is >N—X, where X is H, straight or branched substituted or unsubstituted alkyl, alkenyl or alkynyl, or acyl, carbamoyl, cycloalkyl, aryl, heterocycloalkyl, heteroaryl, aralkyl, amino, alkyl amino, or dialkyl amino; wherein in the compound Z is O or >N—X, where X is H, straight or branched alkyl, alkenyl or alkynyl, or acyl, carbamoyl, cycloalkyl, aryl, heterocycloalkyl, heteroaryl, aralkyl, amino, alkyl amino, or dialkyl amino; wherein each of R₁ and R₂ is H or R₁ and R₂ together are ═O; wherein each of R₃ and R₄ is H or R₃ and R₄ together are ═O; wherein each of R₅ and R₆ are, independently, H, alkyl, or aralkyl; wherein each of R₇ and R₈ is, independently, H, OH or OR₁₄, where R₁₄ is alkyl or —C(O)—R₁₅, where R₁₅ is H, —CH₂R₁₆, —CHR₁₆R₁₆, —CR₁₆R₁₇R₁₆, —OR₁₆, cycloalkyl, aryl, or aralkyl, wherein each R₁₆ is alkyl, cycloalkyl, or aryl, aralkyl; and wherein R₁₇ is alkyl, cycloalkyl, aryl, or aralkyl, or wherein R₇ and R₉ together with the carbons to which each is attached form an oxirane moiety; wherein each of R₉ and R₁₀ is, independently, H, alkyl, OH, or OR₁₃, where R₁₃ is an alkyl, an acyl, or an amide, or R₉ and R₁₀ together are ═CH₂, or wherein R₈ and R₁₀ together with the carbons to which each is attached form an oxirane moiety; wherein if one of R₇ or R⁸ and one of R₉ or R₁₀ is absent, a double bond is formed as indicated by the broken line; and wherein each of R₁₁ and R₁₂ is, independently, H, OH, or OR₁₃, where R₁₃ is an alkyl, an acyl, or an amide, or R₁₁ and R₁₂ together are ═O, or wherein R₁₂ and R₁₀ together with the carbons to which each is attached form an oxetane moiety.

This invention further provides the instant composition, wherein the compound has the structure:

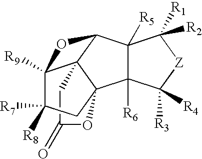

wherein Z is O; wherein each of R₁ and R₂ is H, or R₁ and R₂ together are ═O; wherein each of R₃ and R₄ is H, or R₃ and R₄ together are ═O; wherein each of R₅ and R₆ are, independently, H, alkyl, aralkyl, or aryl; wherein each of R₇ and R₈ is, independently, H, OH or OR₁₄, where R₁₄ is alkyl or —C(O)—R₁₅, where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino, wherein each R₁₆ is straight or branched, substituted or unsubstituted alkyl, alkenyl or alkynyl, cycloalkyl, aryl, heterocycloalkyl, heteroaryl, aralkyl, or amino; and wherein R₁₇ is straight or branched, unsubstituted alkyl, alkenyl or alkynyl, cycloalkyl, aryl, heterocycloalkyl, heteroaryl, aralkyl, or amino; and wherein R₉ is H, alkyl, OH, or OR₁₃, where R₁₃ is an alkyl, an acyl, or an amide.

This invention further provides the instant composition, wherein in the compound R₉ is H, alkyl or OR₁₃, where R₁₃ is an alkyl, an acyl, or an amide. This invention further provides the instant composition, wherein in the compound R₁ and R₂ together are ═O; each of R₃ and R₄ is H; each of R₅ and R₆ are, independently, H, alkyl, or aralkyl; each of R₇ and R₈ is, independently, H, OH or OR₁₄, where R₁₄ is alkyl or —C(O)—R₁₅, where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino, wherein each R₁₆ is straight or branched, substituted or unsubstituted alkyl, alkenyl or alkynyl, cycloalkyl, aryl, heterocycloalkyl, heteroaryl, aralkyl, or amino; and wherein R₁₇ is straight or branched, unsubstituted alkyl, alkenyl or alkynyl, cycloalkyl, aryl, heterocycloalkyl, heteroaryl, aralkyl, or amino; and R₉ is alkyl.

This invention further provides the instant composition, wherein the composition is enantioenriched with an enantiomer having the structure:

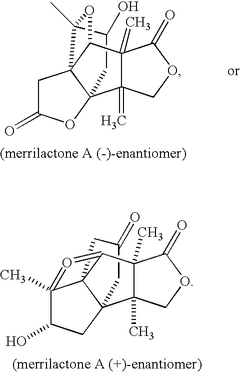

This invention further provides the instant compositions, wherein the compositions are free of plant extracts.

This invention also provides a composition comprising an enantiopure compound free of plant extracts having the structure:

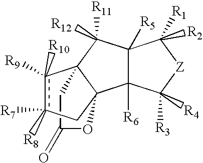

wherein Z is O or >N—X, where X is H, straight or branched substituted or unsubstituted alkyl, alkenyl or alkynyl, or acyl, carbamoyl, cycloalkyl, aryl, heterocycloalkyl, heteroaryl, aralkyl, amino, alkyl amino, or dialkyl amino; wherein each of R₁ and R₂ is H or R₁ and R₂ together are ═O; wherein each of R₃ and R₄ is H or R₃ and R₄ together are ═O; wherein each of R₅ and R₆ are, independently, H, alkyl, aralkyl, or aryl; wherein each of R₇ and R₈ is, independently, H, OH or OR₁₄, where R₁₄ is alkyl or —C(O)—R₁₅, where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino, wherein each R₁₆ is straight or branched, substituted or unsubstituted alkyl, alkenyl or alkynyl, cycloalkyl, aryl, heterocycloalkyl, heteroaryl, aralkyl, or amino; and wherein R₁₇ is straight or branched, unsubstituted alkyl, alkenyl or alkynyl, cycloalkyl, aryl, heterocycloalkyl, heteroaryl, aralkyl, or amino, or wherein R₇ and R₉ together together with the carbons to which each is attached form an oxirane moiety; wherein each of R₉ and R₁₀ is, independently, H, alkyl, OH, or OR₁₃, where R₁₃ is an alkyl, an acyl, or an amide, or R₉ and R₁₀ together are ═CH₂, or wherein R⁸ and R₁₀ together with the carbons to which each is attached form an oxirane moiety; wherein if one of R₇ or R₈ and one of R₉ or R₁₀ is absent, a double bond is formed as indicated by the broken line; and wherein each of R₁₁ and R₁₂ is, independently, H, OH, or OR₁₃, where R₁₃ is an alkyl, an acyl, or an amide, or R₁₁ and R₁₂ together are ═O, or wherein R₁₂ and R₁₀ together with the carbons to which each is attached form an oxetane moiety, or an enantiomer, tautomer or salt of the compound.

This invention further provides the instant composition, wherein Z is >N—X, where X is H, straight or branched substituted or unsubstituted alkyl, alkenyl or alkynyl, or acyl, carbamoyl, cycloalkyl, aryl, heterocycloalkyl, heteroaryl, aralkyl, amino, alkyl amino, or dialkyl amino; or wherein Z is O or >N—X, where X is H, straight or branched alkyl, alkenyl or alkynyl, or acyl, carbamoyl, cycloalkyl, aryl, heterocycloalkyl, heteroaryl, aralkyl, amino, alkyl amino, or dialkyl amino; wherein each of R₁ and R₂ is H or R₁ and R₂ together are ═O; wherein each of R₃ and R₄ is H or R₃ and R₄ together are ═O; wherein each of R₅ and R₆ are, independently, H, alkyl, or aralkyl; wherein each of R₇ and R₈ is, independently, H, OH or OR₁₄, where R₁₄ is alkyl or —C(O)—R₁₅, where R₁₅ is H, —CH₂R₁₆, —CHR₁₆R₁₆, —CR₁₆R₁₇R₁₆, —OR₁₆, cycloalkyl, aryl, or aralkyl, wherein each R₁₆ is alkyl, cycloalkyl, or aryl, aralkyl; and wherein R₁₇ is alkyl, cycloalkyl, aryl, or aralkyl, or wherein R₇ and R₉ together with the carbons to which each is attached form an oxirane moiety; wherein each of R₉ and R₁₀ is, independently, H, alkyl, OH, or OR₁₃, where R₁₃ is an alkyl, an acyl, or an amide, or R₉ and R₁₀ together are ═CH₂, or wherein R₉ and R₁₀ together with the carbons to which each is attached form an oxirane moiety; wherein if one of R₇ or R₈ and one of R₉ or R₁₀ is absent, a double bond is formed as indicated by the broken line; and wherein each of R₁₁ and R₁₂ is, independently, H, OH, or OR₁₃, where R₁₃ is an alkyl, an acyl, or an amide, or R₁₁ and R₁₂ together are ═O, or wherein R₁₂ and R₂₀ together with the carbons to which each is attached form an oxetane moiety.

This invention further provides the instant composition having the structure:

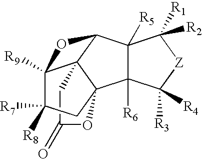

wherein Z is O; wherein each of R₁ and R₂ is H, or R₁ and R₂ together are ═O; wherein each of R₃ and R₄ is H, or R₃ and R₄ together are ═O; wherein each of R₅ and R₆ are, independently, H, alkyl, aralkyl, or aryl; wherein each of R₇ and R₈ is, independently, H, OH or OR₁₄, where R₁₄ is alkyl or —C(O)—R₁₅, where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino, wherein each R₁₆ is straight or branched, substituted or unsubstituted alkyl, alkenyl or alkynyl, cycloalkyl, aryl, heterocycloalkyl, heteroaryl, aralkyl, or amino; and wherein R₁₇ is straight or branched, unsubstituted alkyl, alkenyl or alkynyl, cycloalkyl, aryl, heterocycloalkyl, heteroaryl, aralkyl, or amino; and wherein R₉ is H, alkyl, OH, or OR₁₃, where R₁₃ is an alkyl, an acyl, or an amide.

This invention further provides the instant composition, wherein R₉ is H, alkyl or OR₁₃, where R₁₃ is an alkyl, an acyl, or an amide; or wherein R₁ and R₂ together are ═O; wherein each of R₃ and R₄ is H; wherein each of R₅ and R₆ are, independently, H, alkyl, or aralkyl; wherein each of R₇ and R₈ is, independently, H, OH or OR₁₄, where R₁₄ is alkyl or —C(O)—R₁₅, where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino, wherein each R₁₆ is straight or branched, substituted or unsubstituted alkyl, alkenyl or alkynyl, cycloalkyl, aryl, heterocycloalkyl, heteroaryl, aralkyl, or amino; and wherein R₁₇ is straight or branched, unsubstituted alkyl, alkenyl or alkynyl, cycloalkyl, aryl, heterocycloalkyl, heteroaryl, aralkyl, or amino; and wherein R₉ is alkyl.

This invention further provides the instant composition, wherein the enantiopure compound has the structure:

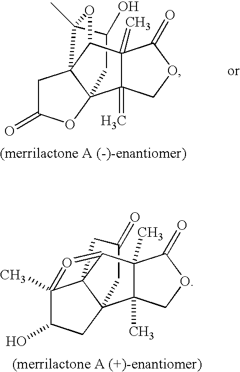

This invention also provides a process for preparing a composition enantioenriched with a (+)-enantiomer or a (−) -enantiomer of merrilactone A, and optionally purifying the (+)-enantiomer or the (−)-enantiomer of the merrilactone A to produce enantiopure merrilactone A comprising:

reacting

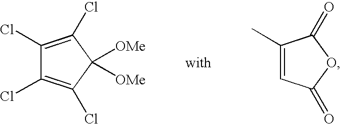

at a temperature of 140° C. to 230° C. to produce a compound having the structure:

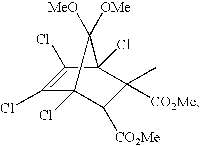

b) stereospecifically C-methylating the compound produced in step a) to produce a compound having the structure:

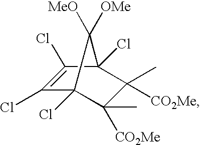

c) treating the compound produced in step b) with a suitable source of hydride and refluxing, and then with Na, NH₃ or Na, EtOH or Li, NH₃ to producea compound having the structure:

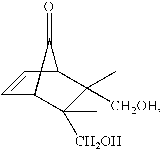

d) treating the compound produced in step c) with 2,2-dimethoxypropane, acetone and pTsOH, then treating the compound with NaH, (EtO)₂POCH₂CO₂Et, and THF, and then treating the compound with Mg and MeOH to produce a compound having the structure:

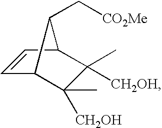

e) treating the compound produced in step d) with dimethyldioxirane and CH₂Cl₂ to give a compound having the structure:

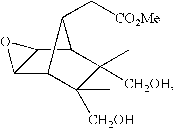

f) exposing the compound produced in step e) to either (S,S)-[CoIII(salen)]-OAc or (R,R)-[CoIII(salen)]-OAc at −110° C. to −55° C., and then to THF −45° C. to −5° C. to give an enantiomeric enriched compound having the structure:

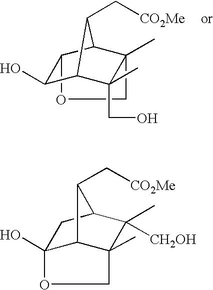

g) oxidizing the compound produced in step f) with PDC and DMF and then esterifying the product with K₂CO₃, MeI and acetone to give a compound having the structure:

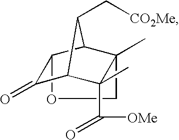

h) treating the compound produced in step g) with magnesium monoperoxyphthalate hexahydrate and MeOH at −10° C. to +10° C. to produce a compound having the structure:

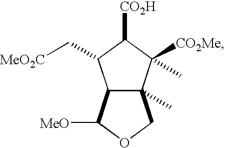

i) treating the compound produced in step h) with DCC and mCPBA at −10° C. to +10° C., and then refluxing the compound with PhH, and then treating the compound with and MeOH to produce a compound having the structure:

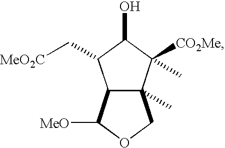

j) treating the compound produced in step i) with BF₃.OEt₂, or TiCl₄ or PTsOH to produce a compound having the structure:

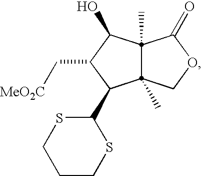

k) treating the compound produced in step j) with and CH₃CN/H₂O, and then with NaBH₄ and MeOH at −10° C. to +10° C., to produce a compound having the structure:

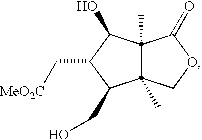

l) treating the compound produced in step k) with o-NO₂C₆H₄SeCN, Bu₃P, and THF, then 25%-35% H₂O₂, then treating the compound with a silyl protecting group, Et₃N and CH₂Cl₂ to produce a compound having the structure:

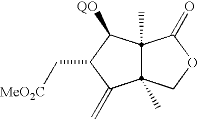

where Q is a silyl protecting group,
m) treating the product of step l) with LiOH, MeOH/H₂O and then I₂ in saturated NaHCO₃/THF, a compound having the structure:

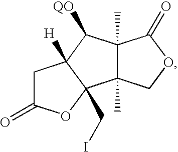

n) processing the product of step m) to produce the composition enantioenriched with a (+)-enantiomer or a (−) -enantiomer of the merrilactone A, and optionally purifying the (+)-enantiomer or a (−)-enantiomer of the merrilactone A to produce the enantiopure merrilactone A.

This invention further provides the instant process, wherein in step c) the source of hydride is LAH and THF; wherein in step n) the optical purification of (+)-enantiomer or (−)-enantiomer of the merrilactone A is effected by recrystallization; wherein the produced composition is enantioenriched with the (−)-enantiomer and the catalyst in step f) is (S,S)-[Co^{III}(salen)]-OAc; wherein the produced composition is enantioenriched with the (+)-enantiomer and the catalyst in step f) is (R,R)-[Co^{III}(salen)]-OAc.

In one embodiment of the instant process, step a) comprises treating in the presence of MeOH, then refluxing, then treating in the presence PhH-Me-OH then TMSCHN₂. In one embodiment of the instant process the compound in step b) is stereospecifically C-methylated using LDA, HMPA, MeI, and THF at −110° C. to −55° C. In one embodiment of the instant process the oxidizing in step d) is performed using mCPBA and CH₂Cl₂. In one embodiment of the instant process the product of step e) is treated in step f) with dimethyldioxirane in CH₂Cl₂.

This invention provides a process, wherein when producing the enantioenriched composition in step n) comprises:
a) treating the product of step m) of the instant process with allylSnBu₃ to produce a compound having the structure:

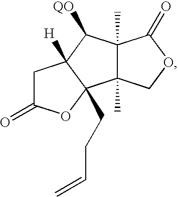

b) treating the product of step b) with LHMDS, and PhSeCl, and then with PhSeBr and MeCN to produce a compound having the structure:

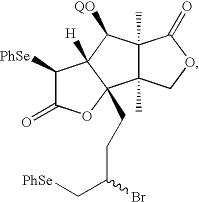

c) treating the product of step b) with O₃, CH₂Cl₂ and 1-hexene to produce a compound having the structure:

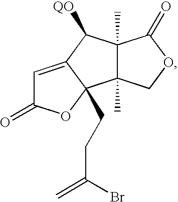

d) treating the product of step c) with Bu₃SnH and AlBN to produce a compound having the structure:

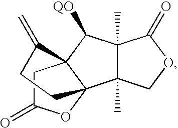

e) treating the product of step d) with TsOH to produce a compound having the structure:

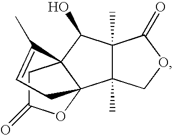

f) treating the product of step e) with mCPBA or a dimethyldioxirane to produce a compound having the structure:

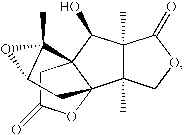

g) treating the product of step f) with an acid to produce the composition enantioenriched with the (+)-enantiomer or the (−)-enantiomer of the merrilactone A.

This invention also provides a process for preparing a racemic composition comprising an equimolar mixture of a pair of enantiomers having the structures:

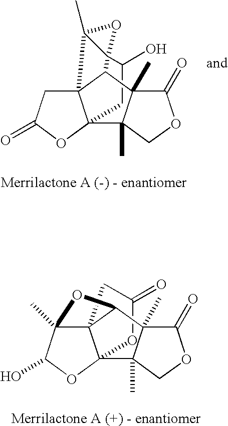

comprising:
a) reacting

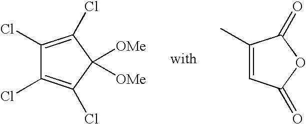

at a temperature of 140° C. to 230° C. to produce a compound having the structure:

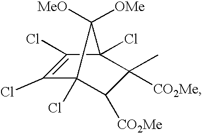

b) stereospecifically C-methylating the compound produced in step a) to produce a compound having the structure:

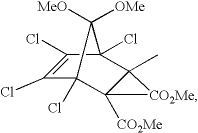

c) treating the compound produced in step b) with a suitable source of hydride and refluxing, and then with Na, NH₃ or Na, EtOH or L₁, NH₃ to produce a compound having the structure:

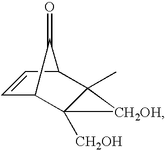

d) treating the compound produced in step c) with 2,2-dimethoxypropane, acetone and pTsOH, then treating the compound with NaH, (EtO)₂POCHzCO₂Et, and THF, and then treating the compound with Mg and MeOH to produce a compound having the structure:

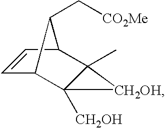

e) oxidizing the compound produced in step d) to produce a compound having the structure:

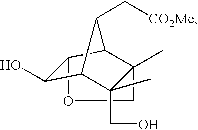

f) oxidizing the compound produced in step e) with PDC and DMF and then esterifying the product with K₂CO₃, MeI and acetone to give a compound having the structure:

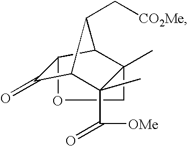

g) oxidizing the compound produced in step f) with magnesium monoperoxyphthalate hexahydrate and MeOH at −10° C. to +10° C. to produce a compound having the structure:

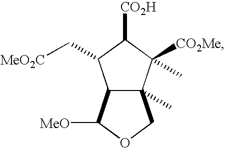

h) treating the compound produced in step g) with DCC and mCPBA at −10° C. to +10° C., and then treating the product with PhH, and then treating the product with K₂CO₃ and MeOH to produce a compound having the structure:

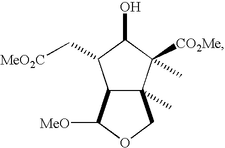

i) treating the compound produced in step h) with BF₃.OEt₂, or TiCl₄ or PTsOH to produce a compound having the structure:

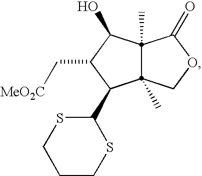

j) treating the compound produced in step i) with PhI (OCF₃CO₂)₂ and CH₃CN/H₂O, and then with NaBH₄ and MeOH at −10° C. to +10° C. to produce a compound having the structure:

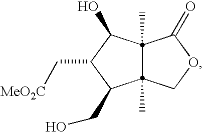

k) treating the compound produced in step j) with o-NO₂C₆H₄SeCN, Bu₃P, and THF, then 25%-35% H₂O₂, then treating the compound with a silyl protecting group, Et₃N and CH₂Cl₂ to produce a compound having the structure:

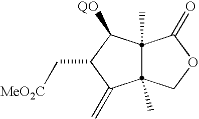

where Q is a silyl protecting group,
l) treating the product of step k) to LiOH, MeOH/H₂O and then I₂ to produce a compound having the structure:

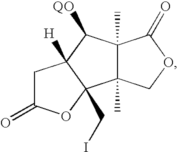

m) processing the product of step 1) to produce the racemic composition.

In one embodiment of the instant process, in step c) the suitable source of hydride is LAH and THF. In one embodiment of the instant process m) comprises:
a) treating the product of step l) with allylSnBu₃ to produce a compound having the structure:

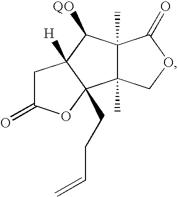

b) treating the product of step a) with LHMDS, TMSCl and PhSeCl, and then with PhSeBr and MeCN to produce a compound having the structure:

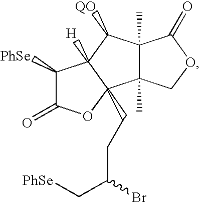

c) treating the product of step b) with O₃, CH₂Cl₂ and 1-hexene to produce a compound having the structure:

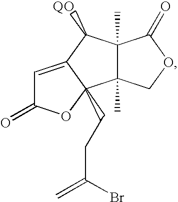

d) treating the product of step c) with Bu₃SnH and AIBN to produce a compound having the structure:

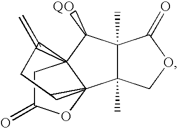

e) treating the product of step d) with TsOH to produce a compound having the structure:

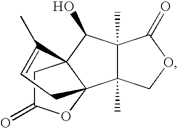

f) treating the product of step e) with mCPBA or a dimethyldioxirane to produce a compound having the structure:

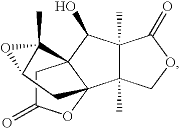

g) treating the product of step f) with an acid to produce the composition.

In one embodiment of the instant process, step a) comprises treating in the presence of MeOH, then refluxing, then treating in the presence PhH-Me-OH then TMSCHN₂.

In one embodiment of the instant process the compound in step b) is stereospecifically C-methylated using LDA, HMPA, MeI, and THF at −110° C. to −55° C.

In one embodiment of the instant process the oxidizing in step d) is performed using mCPBA and CH₂Cl₂.

In one embodiment of the instant process the product of step e) is treated in step f) with dimethyldioxirane in CH₂Cl₂.

This invention also provides a compound having the structure:

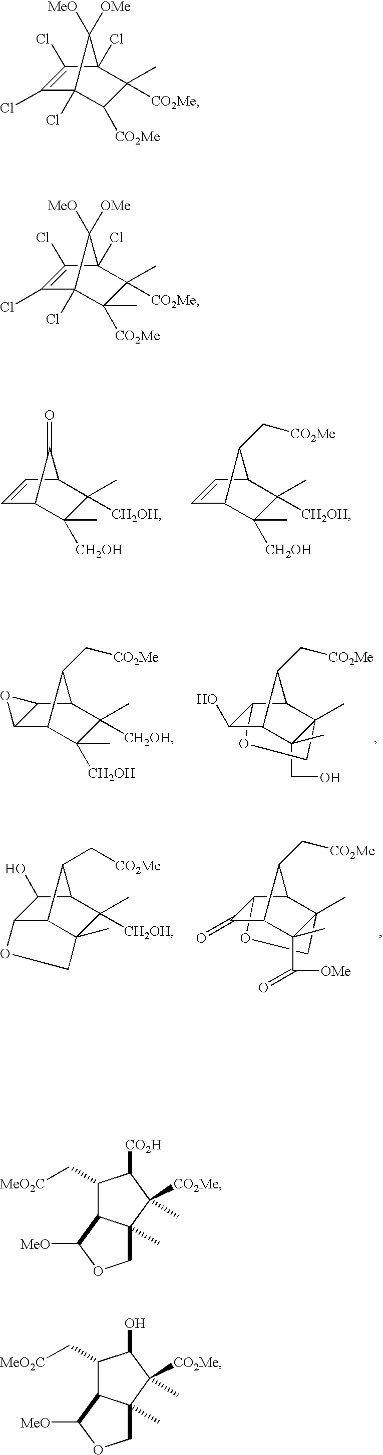

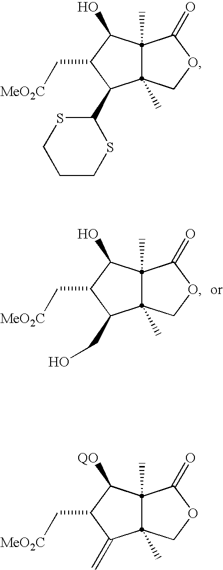

where Q is a silyl protecting group,
   and enantiomers thereof.

This invention also provides a method of alleviating a side effect resulting from a therapy-induced neuropathy in a patient receiving the therapy comprising administering to the patient any one of the instant compositions in an amount effective to alleviate the side effect. In one embodiment the therapy is a chemotherapy. In embodiments the chemotherapy comprises administering Arsenic trioxide, Alemtuzumab, Bortezomib, Altretamine, Docetaxel, Capecitabine, Oxaliplatin, Carboplatin, Paclitaxel, Cisplatin, Thalidomide, Dacarbazine, Denileukin diftitox, Fludarabine Interferon alpha, Liposomal daunorubicin, Tretinoin, Vinblastine, Vinorelbine, Vincristine.

In one embodiment the side effect is tingling sensation or numbness in hands, feet, or limbs. In one embodiment the side effect is a peripheral neuropathy.

This invention also provides a method of treating a peripheral neuropathy in a patient suffering from a diabetes comprising administering to the patient any one of the instant compositions in an amount effective to treat the peripheral neuropathy.

This invention also provides a method of treating a peripheral neuropathy in a patient suffering therefrom comprising administering to the patient any one of the instant compositions in an amount effective to treat the peripheral neuropathy.

The abbreviations used are defined below:

THF=tetrahydrofuran
TBS=tert-butyldimethylsilyl
PhH=benzene
MeOH=methanol
mCPBA=meta-chloroperbenzoic acid
pTsOH=para-toluenesulfonic acid
PhI(OCF₃CO₂)₂—[Bistrifluoroacetoxy)-iodo]benzene
Bu₃P—tributyl phosphine
allylSnBu₃—allyltributyltin
LHMDS—lithium bis(trimethylsilyl)amide
PhSeCl—phenyl selenenyl chloride
PhSeBr—phenyl selenenyl bromide

### DMF—N,N-dimethylformamide

### DCC—N,N-dicyclohexylcarbodiimide

DMDO—2,2-dimethyldioirane
HMPA—hexamethylphosphoramide
LAH—lithium aluminium hyride
LDA—lithium diisopropylamide
mCPBA—meta-chloroperoxybenzoic acid
MMPP=magnesium monoperoxyphthalate
salen—N,N′-bis(salicyldiene)ethylenediamine
TBS—tert-butyl dimethylsilyl
TMS—trimethylsilyl
Ts—toluenesulfonyl
Tf—trifluoromethanesulfonyl
PDC—pyridinium dichromate

“Free of plant extract” as used here means absent of any amount of Illicium plant species-specific materials, such as anislactones. Thus only synthetically produced compositions could be free of plant extract. Any compositions isolated from a plant would always contain at least some trace amount of plant material.

“Enantioenriched composition” as used here means a composition of a chiral substance whose enantiomeric ratio is greater than 50:50 but less than 100:0. (See IUPAC Compendium of Chemical Terminology, “Goldbook”, Second Edition, 1997).

“Enantiopure composition” as used herein means a composition containing molecules all having the same chirality sense (within the limits of detection). (See IUPAC Compendium of Chemical Terminology, “Goldbook”, Second Edition, 1997).

“Racemic mixture”, “racemic composition”, “racemic”, “racemate” and “(±)” terminology are used interchangeably herein.

The invention further contemplates the use of prodrugs which are converted in vivo to the compounds of the invention (see, e.g., R. B. Silverman, 1992, “The Organic Chemistry of Drug Design and Drug Action”, Academic Press, Chapter 8, the entire contents of which are hereby incorporated by reference). Such prodrugs can be used to alter the biodistribution (e.g., to allow compounds which would not typically enter a reactive site) or the pharmacokinetics of the compound.

Certain embodiments of the disclosed compounds can contain a basic functional group, such as amino or alkylamino, and are thus capable of forming pharmaceutically acceptable salts with pharmaceutically acceptable acids, or contain an acidic functional group and are thus capable of forming pharmaceutically acceptable salts with bases. The instant compounds may be in a salt form. As used herein, a “salt” is salt of the instant compounds which has been modified by making acid or base salts of the compounds. In the case of compounds used for treatment of cancer, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. For example, an amount effective to inhibit or reverse neurite damage, or for example to inhibit, attenuate or reverse neurodegenerative disorder symptoms. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

The merrilactones and analogs thereof of this invention are useful as neurotrophic factors and may be employed in treatment of neuropathies and other nerve-related damage.

In addition, they may be employed as adjuncts in therapies that can cause neurological impairment, such as with certain anti-cancer agents that cause “tingling” sensations due to neurite damage, or as with diabetic patients. Thus, the merrilactones and analogs thereof of this invention may be administered alone or in combination with chemotherapies to patients in need thereof, or for example to diabetic patients, in order to provide symptomatic relief. The compositions of this invention may be administered in various forms, including those detailed herein. As used herein, “treatment” of a neurite damaging disorder, or another neurodegenerative disorder encompasses inducing inhibition, regression, or stasis/prevention of the disorder. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed. The treatment may also be an adjunct to another therapy, e.g. chemotherapy, which itself causes the neurite damage. Both iatrogenic and naturally occurring neurite damage may be treated with the compounds of this invention.

In this regard, it is noted that some chemotherapy drugs can cause a side effect of peripheral neuropathy. Currently, there is little that can be done to reduce the risks of neuropathy in patients undergoing chemotherapy, particularly when platinum complexes are involved. A peripheral neuropathy results in damage to the nerves between the extremities and the central nervous system (CNS). If steps are not taken, peripheral neuropathy can become a long-term problem for patients receiving chemotherapy. For a patient suffering such a peripheral neuropathy, sensations of numbness and tingling of extremities (e.g. hands and feet) are common. In addition, peripheral neuropathy may also be mediated by a number of other diseases and conditions. Some of the causes include alcoholism, diabetes mellitus, certain B-vitamin deficiencies, inherited conditions, and others. Many of these neuropathies are reversible if treated promptly.

“Peripheral neuropathy” as used herein, refers to abnormal function or pathological changes in nerves located outside of the brain or spinal column. The nerves may be sensory, motor, sensorimotor or autonomic and dysfunction may manifest itself in any of the various symptoms discussed herein. The peripheral neuropathy may be iatrogenic or may be naturally occurring alone or as a secondary effect of a primary disease.

“Treatment” of a peripheral neuropathy as used herein shall include ameliorating, slowing, stopping or reversing the peripheral neuropathy and/or ameliorating or alleviating symptoms associated with the peripheral neuropathy including numbness or tingling in a patient's extremities.

“Chemotherapy” as used herein shall mean the use of chemical agents in the treatment or control of disease, such as a cancer.

“Therapy-induced neuropathy” shall mean peripheral neuropathies induced by medical treatment, i.e. iatrogenic peripheral neuropathies. Examples include peripheral neuropathies induced by chemotherapy.

U.S. Pat. No. 6,743,824 which discusses peripheral neuropathies and their treatment, including peripheral neuropathies induced by drugs and U.S. Pat. No. 6,075,053 which discusses reversal or treatment of neuropathy, are both hereby incorporated by reference.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutical carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with anti-cancer compounds, or tumor growth inhibiting compounds, or with other compounds also used to treat neurite damage. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the cancer, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The instant compounds may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The present invention also includes pharmaceutical kits useful, for example, for the treatment of neurodegenerative disorders or neurite damage, or neurite damage associated with anti-cancer therapies or other therapies, which comprise one or more containers containing a pharmaceutical composition comprising an effective amount of one or more of the compounds. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement. As used herein, “alkyl” means C₁-Cₙ, and is defined to include groups having 1, 2, 3, 4, 5, 6 etc. carbons in a linear or branched arrangement, and specifically includes methyl, ethyl, propyl, butyl, pentyl, hexyl, and so on. “Alkyl” in regard to any of R¹ through R¹² as used here is C₁-Cₙ. “Alkoxy” represents an alkyl group of indicated number of carbon atoms attached through an oxygen bridge.

The term “alkyl” as used in the terms “-alkyl-OH”, “—NH-alkyl”, “-alkyl- (NH₂)”, “-alkyl-C(O) (OH”, and “—O-alkyl” are C₁-Cₙ alkyl as defined above, i.e. they include groups having 1, 2, 3, 4, 5, or n carbons in a linear or branched arrangement. For example methyl, ethyl, propyl, butyl, pentyl, or hexyl in a linear or branched arrangement.

The term “alkyl” as used in the term “—N(alkyl)₂” means C₁-Cₙ alkyl as defined above, i.e. they include groups having 1, 2, 3, 4, 5, or n carbons in a linear or branched arrangement. However, the two alkyl groups of “—N(alkyl)₂” need not necessarily be the same type of alkyl group. For example one alkyl may be chosen from the group methyl, ethyl, propyl, butyl, pentyl, or hexyl in a linear or branched arrangement and the other alkyl may be independently chosen from the group methyl, ethyl, propyl, butyl, pentyl, or hexyl.

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing at least 1 carbon to carbon double bond, and up to the maximum possible number of non-aromatic carbon-carbon double bonds may be present. For example, “C₂-C₆ alkenyl” means an alkenyl radical having 2, 3, 4, 5, or 6 carbon atoms, and at least 1 carbon-carbon double bond, and up to, for example, 5 carbon-carbon double bonds in the case of a CG alkenyl. respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. “Alkenyl” with regard to R¹ through R¹² as used here is C₂-Cₙ.

The term “cycloalkenyl” shall mean cyclic rings of 3 to 10 carbon atoms and at least 1 carbon to carbon double bond (i.e., cycloprenpyl, cyclobutenyl, cyclopenentyl, cyclohexenyl, cycloheptenyl or cycloocentyl).

The term “alkynyl” refers to a hydrocarbon radical straight or branched, containing at least 1 carbon to carbon triple bond, and up to the maximum possible number of non-aromatic carbon-carbon triple bonds may be present. Thus, “C₂-C₆ alkynyl” means an alkynyl radical radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. “Alkynyl”, with regard to R¹ through R¹² as used here is C₂-Cₙ.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

The term “heteroaryl”, as used herein, represents a stable monocyclic or bicyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo”, “halide”, or “halogen” as used herein is intended to include chloro, fluoro, bromo and iodo.

The term “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

The alkyl, alkenyl, alkynyl, cycloalkyl, aryl, heteroaryl and heterocyclyl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, alkynyl, cycloalkyl, cycloalkenyl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing compounds of the present invention, one of ordinary skill in the art will recognize that the various substituents, i.e. R¹ through R¹² are to be chosen in conformity with well-known principles of chemical structure connectivity.

The merrilactone analogs produced here may be made directly from starting products or can be made from the merrilactone A enantiomers or racemic mixtures disclosed here.

All combinations of the various elements are within the scope of the invention

### EXPERIMENTAL RESULTS

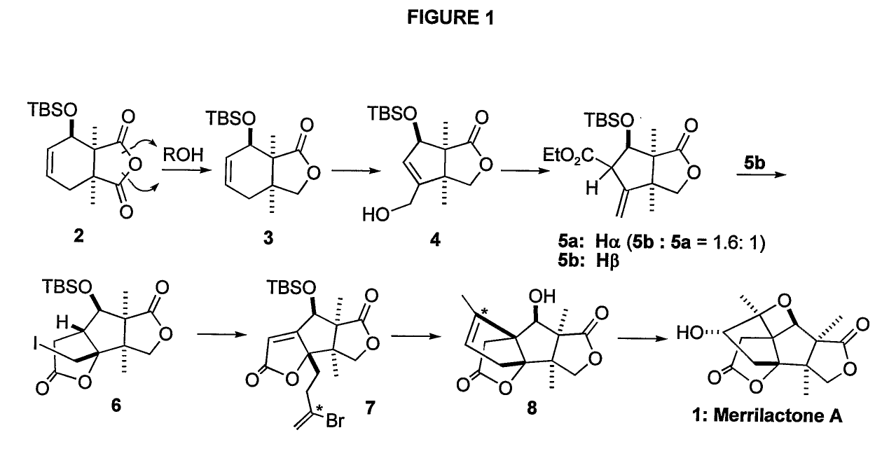

As shown in FIG. 1, (the Birman-Danishefsky), chirality is initially introduced in our first generation synthesis in the context of the Diels-Alder reaction leading to 2. Since this reaction can only be accomplished at high temperatures, the prospects for strong margins of catalytically mediated enantioselection are not promising. These considerations, particularly the goal of generating the enantiopure antipodes of merrilactone A for biological assessment, led us to explore a new total synthesis route. Since the previous route to merrilactone A from 6 onward is rather concise and efficient, we planned for this compound to be a milestone in a new route.

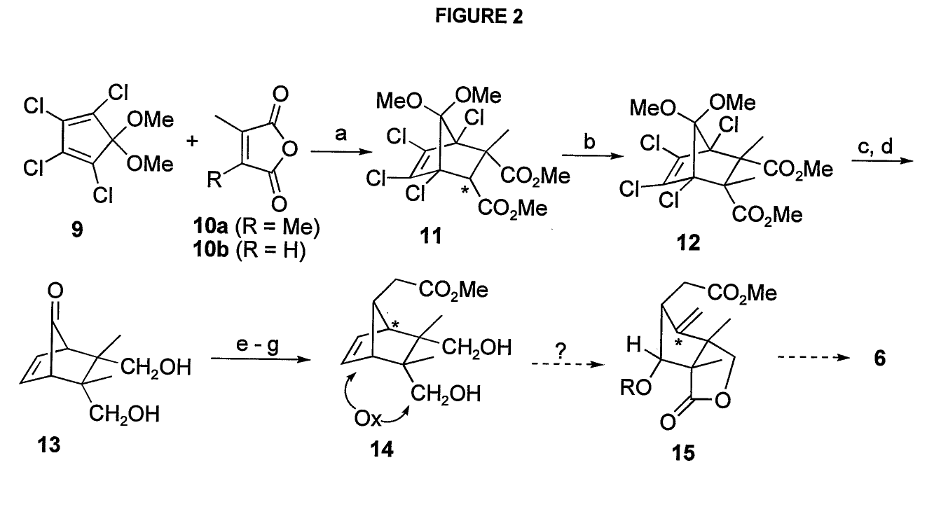

Toward this end, diester compound 12 was synthesized. It could not be reached by Diels-Alder reaction of 10a (R=Me) with 9. A solution around this problem was required. Fortunately, cycloaddition could be accomplished using the monomethyl compound 10b (R═H) with endo specificity (FIG. 2). Methanolysis of the anhydride and esterification of the free acid, as shown, afforded 11. The key point was that the quaternary ester in 11 was endo. As shown, lithiation generated the enolate of the other ester (see asterisk in 11). In the event, stereospecific C-methylation of this enolate gave rise to 12. The latter was advanced by the straightforward steps shown, to meso structure 14.

At this stage, the global assignment was that of accomplishing the degradation of 14 to reach 15. The latter would intersect 6 by iodolactonization. Overall, we had to accomplish oxidation of the 1,4-diol of 14 to a butyrolactone and degradation of the etheno linkage with interpolation of an oxygen at one erstwhile bridgehead center and attachment of an exocyclic methylene to the other (see asterisks in 14 and 15). The crux of the more subtle challenge of specificity at the regiochemical level is that the interpolated oxygen appear “ortho” to the oxidized carbon of the lactone, leaving the exo methylene group to emerge ortho to the unoxidized hydroxymethyl equivalent (see structure 15).

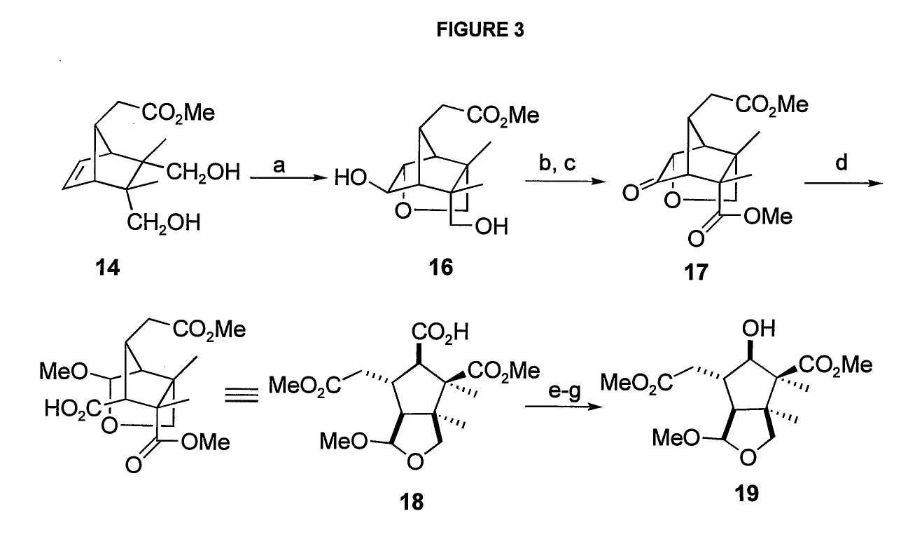

In the event, oxidation of 14 with mCPBA resulted in the formation of 16 (FIG. 3). Compound 16 was subjected to PDC oxidation, which, following esterification, led to the formation of ketoester 17. Baeyer-Villiger oxidation of 17 gave rise to 18 (4). The resulting carboxylic acid in 18 was transformed to the requisite secondary alcohol with retention of stereochemistry through “carboxy inversion,” (5) leading to 19.

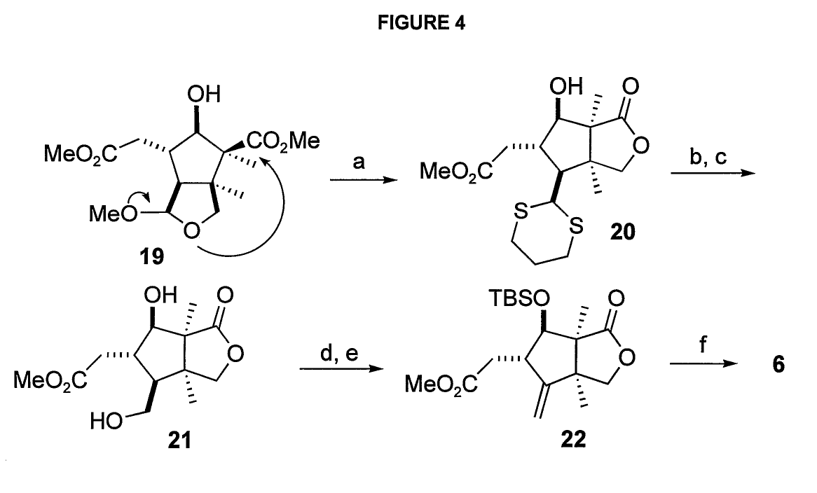

Ring opening of the methoxytetrahydrofuran moiety of 19 was accomplished by trapping of its masked aldehyde, prompting lactonization to produce 20 (see arrows). The latter was subsequently converted to 21 (FIG. 4). In this way, the regiochemical issues delineated above had been settled in a most favorable manner.

Compound 21, upon exposure to the Grieco protocols (6), underwent selective reaction at the primary alcohol to provide a transient selenide, which suffered oxidative elimination to afford the desired exocyclic olefin. Silyl protection of the secondary alcohol gave rise to 22. The latter was hydrolyzed and the resultant carboxylic acid suffered iodolactonization to afford the advanced intermediate 6, whose spectroscopic properties were in complete accord with those previously reported (1).

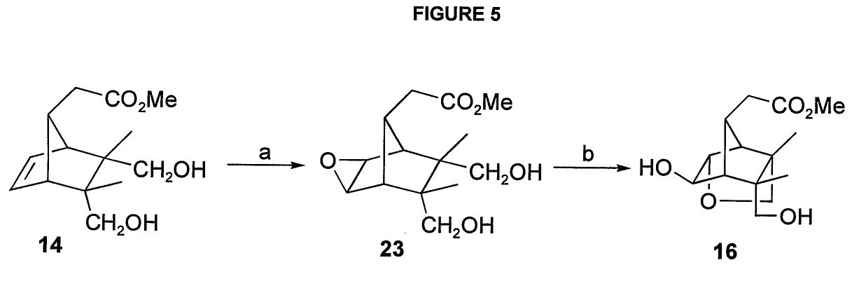

Having achieved a controlled synthesis of 6, an enantioselective synthesis of merrilactone A was pursued The second generation synthesis was built around a series of meso intermediates culminating in 14 and thence its exo epoxide 23 (FIG. 5). It was hoped to make use of Jacobsen's innovative intramolecular asymmetric ring opening (ARO) methodology. In the event, compound 14 was treated with dimethyldioxirane to form the discrete epoxide, 23. The latter was exposed to catalytic amounts of (S,S)-[Co^{III}(salen)] as described by Jacobsen (7) (Scheme 5). We were pleased to find, in practice, that this treatment led to the formation of enantioenriched 16, in 86% ee and 86% yield. Additionally, use of the R,R Jacobsen catalyst led to ent-16.

Compounds 16 and ent-16 were converted into the corresponding benzyl esters, and the optical rotations were determined: [α]²³_{D} −10.9 (CHCl₃, c 0.19) for the benzyl ester of 16 and [α]²³_{D} 7.9 (CHCl₃, c 0.34) for the benzyl ester of ent-16.

One of the key teachings of this synthesis lies in the construction of 12. Thus, we were able to compensate for the reticence of dimethyl maleic anhydride 10a to undergo cycloaddition by performing a series of straightforward transformations with Diels-Alder adduct 11, leading to the formation of 12. A second important feature of this synthesis is the chemical degradation pathway from 14 to 18, which proceeds with full regiocontrol and promising enantiocontrol.

### Asymmetric Synthesis of Merrilactone A

There are several general mechanisms through which to achieve enantiocontrol in total synthesis. Perhaps the most straightforward approach is to design the synthetic route such that one of the starting materials is a member of the chiral pool of readily available, naturally occurring compounds (cf. amino acids, carbohydrates). Alternatively, one might temporarily install a readily removable chiral auxiliary, which would dictate facial selectivity in a key stereodefining transformation. Upon cleavage of the auxiliary, the previous diastereo bias translates to enantio bias. The burgeoning field of enantioselective catalysis relies on catalysts of defined chirality to exert stereofacial control in the transition states of stereodefining reactions. Enantioselective catalytic methods have been developed to introduce chirality to substrates lacking stereocenters and, less frequently, to effect the desymmetrization of meso compounds.

Lipases are one of the most widely used enzymes in asymmetric synthesis. Here, a lipase was applied to enantioselectively differentiate the two hydroxymethyl groups in meso diol via transesterification or hydrolysis of the corresponding bis-acetate derivative. There was no sign of any transesterification for three tested meso diols, and out of three available acetates, only the less hindered acetate was hydrolyzed.

Hayashi's asymmetric hydrosilylation with trichlorosilane provides a useful method for the enantioselective one-pot transformation of an olefin to an alcohol. Here, with Hayashi's method, this conversion was achieved only poorly ˜20% ee.

### Successful Application of Jacobsen's Asymmetric Ring Opening Methodology

The hypothesis made here is that transformation of 14 to 16 would be amenable to enantioselective catalysis. The asymmetric catalytic epoxide ring opening methodology developed by Jacobsen and co-workers was chosen to test this. In 1999, Jacobsen reported that epoxy diol 84 cyclized under the influence of (R,R)-[Co^{III}(salen)]-OAc catalysis to afford the desymmetrized bicyclic ether 85 (scheme 1). More importantly, both (R,R)-[Co^{III}(salen)]-OAc and (S,S)-[Co^{II}(salen)]-OAc catalysts could be easily prepared in large quantities from commercially available (R,R)-Co^{II}(salen) and (S,S)-Co^{II}(salen) respectively.

Intermediate 14 was treated with DMDO to afford the meso epoxide intermediate 23. The reaction conditions were so mild that no epoxide ring opening product was observed in the NMR of the crude product. Epoxy diol 23 was treated with catalytic amounts of (R,R)-[Co^{III}(salen)]-OAc to afford intermediate ent-16 in 85% yield over the two steps. It was found that this reaction proceeded with good levels of enantiocontrol, providing ent-16 in 86% ee. Treatment of 23 with the opposite catalyst enantiomer (S,S)-[Co^{III}(salen)]-OAc provided 16 in the same yield and ee. The absolute configurations of both ent-16 and 16 are made by analogy to Jacobsen's model study. Scheme 1 shows desymmetrization of meso compound by Jacobsen's Intramolecular Asymmetric Ring Opening (ARO).

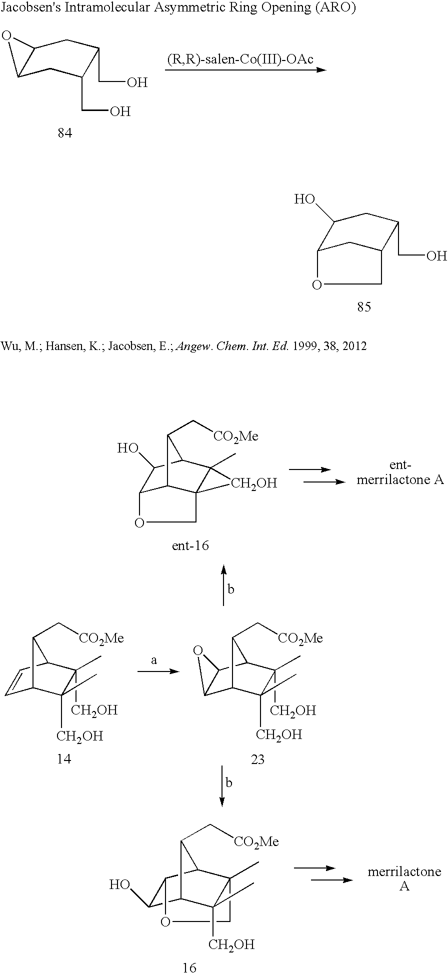

Reagents and Conditions: a) DMDO, CH₂Cl₂, 0.5-1 h; b) (R,R)-[CoIII(salen)]-OAc, −78° C., two days; then −25° C., two days, THF, 86% over two steps; c) (S,S)-[CoIII(salen)]-OAc, −78° C., two days; then −25° C., two days, THF, 85% over two steps (n.b. see below regarding actual determined stereochemistry and Scheme 6).

With both enantioenriched ent-16 and 16 from enantioselective desymmetrization in hands, the route described above was then applied to transform them to the corresponding merrilactone A antipode. The route starting from 16 is described here, but the method can be applied mutatis mutandis to ent-16, with the corresponding stereochemistry changes to the structures.

Cyclic ether 16 could be easily transferred to the enantioriched 29. Chain extension of 29 by the elegant Keck C-allylation method with allyltributyltin gave the required “anti-backbone” isomer 30 (scheme 2).

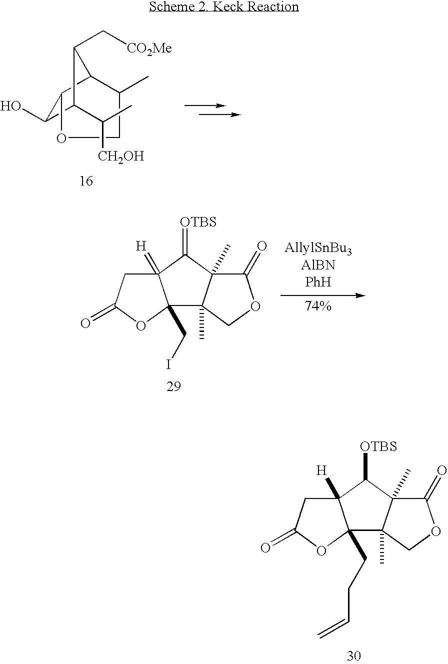

The Birman-Danishefsky key radical cyclization step required conversion of 30 to 14. This was accomplished in 3 steps: (1) selenenation at C10 via reaction of with PhSeCl, and (2) subsequent bromoselenenation of the terminal vinyl group of 29 gave the required bis-seleno intermediate 31, and then (3) concurrent oxidative deselenation afforded the desired 32.

Treatment of 32 under the standard radical cyclization conditions afforded a 90% yield of 33, thereby completing the construction of the carbon skeleton of merrilactone A (scheme 3).

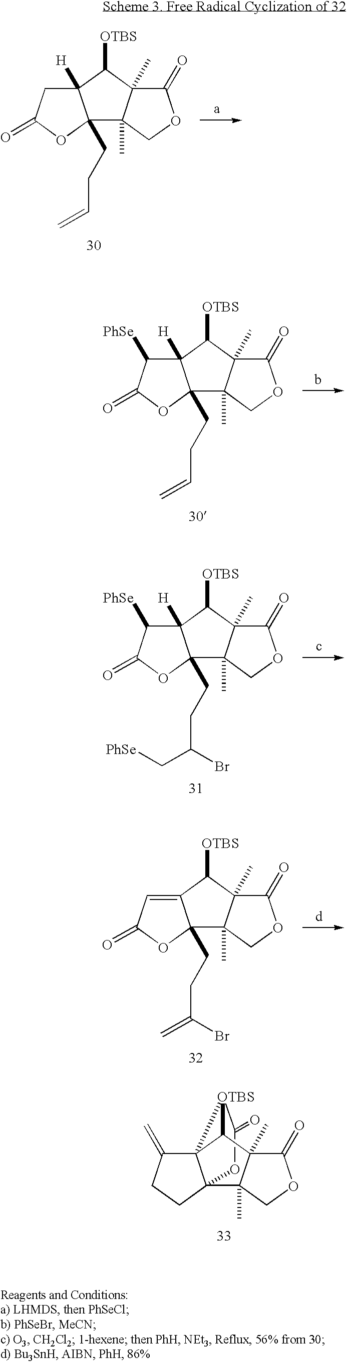

Isomerization of the exo methylene group of 33 was concurrent with liberation of the C7 β-alcohol to provide 34. In Fukuyama's conversion of anislactone B to merrilactone A and the first generation synthesis described earlier, mcPBA was used as the oxidant for the epoxidation reaction. Only moderate selectivity (3.5:1) was achieved. This could be explained as following: while hydroxyl groups have often been used to direct epoxidation with peracids in a syn sense, the congested nature of the β-face of the C₁-C₂ double bond is such that epoxidation occurs primarily (3.5:1) from its α-face. Perhaps if the oxidant was switched to some reagent that was less likely to interact with hydroxy groups, better selectivity would be achieved in the epoxidation step. In the event, it was found that epoxidation of 34 using DMDO generated 35 as the sole product (scheme 4).

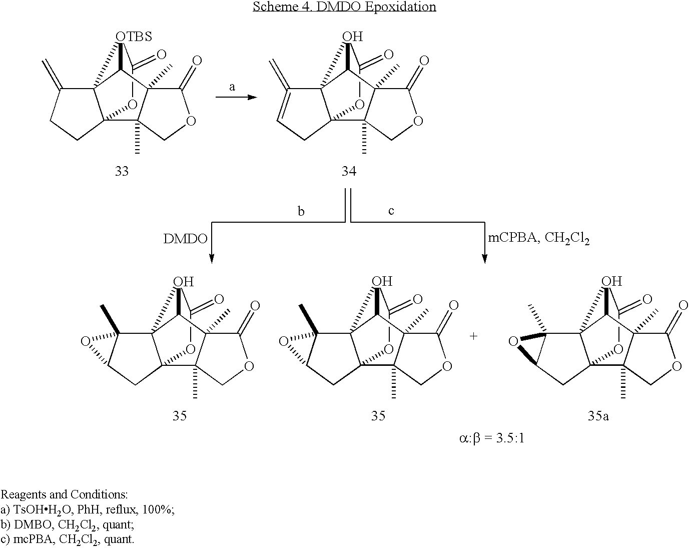

In the final stage of the synthesis, merrilactone A 1 is produced by an acid-induced homo-Payne rearrangement of (scheme 5). The spectroscopic properties of 1 were in complete accord with the published data.

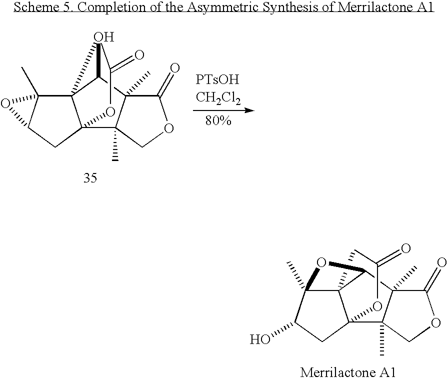

### Optical Analysis of Merrilactone Enantiomers

The reported data for natural merrilactone A is [α]D²¹=+11.8 (c=1.2, MeOH). Surprisingly, we achieved preparation of a product having an optical rotation of about +12 (+10 to +13, MeOH, C=0.15) using (R,R)-[Co^{III}(salen)]-OAc catalyst. The optical rotation of the material prepared using (S,S)-[Co^{III}(salen)]-OAc catalyst is about −12 (−10 to −13 measured, MeOH, C=0.3). Thus, the (S,S) catalyst actually produces ent-merrilactone A and the (R,R) catalyst actually produces merrilactone A.

In explaining these results, it is possible that Jacobsen's assignment of stereochemistry is correct, but it could not be applied to the present case. Based on a comparison to the analogous Jacobsen case B, (R,R)-[Co^{III}(salen)]-OAc should have led to ent-merrilactone A. However, upon comparison with Jacobsen's closest example, it is noted that the present substrate has the bridge, two methyls, and an acetate side chain, and consequently, there might be no direct analogy to Jacobsen's example. From the data provided here, it appears that the chemistry is correct and Fukuyama's analysis is correct, but that there is indeed no direct analogy to the closest Jacobsen example. The stereochemistry is actually as that set forth in scheme 6.

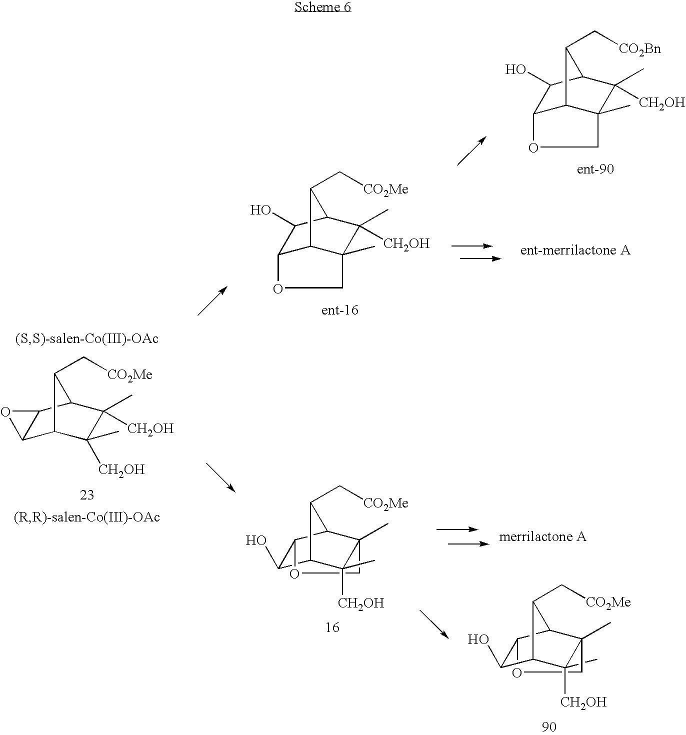

In summary, a route to enantiopure merrilactones has been achieved. Moreover, the chemistry described herein serves to ameliorate the selectivity awkwardness of the earlier method of producing racemic merrilactone A.

### Materials and Methods

All reactions were carried out under an argon atmosphere. Tetrahydrofuran, diethyl ether, and dichloromethane were purified by passing through solvent columns. Other solvents were obtained commercially and were used as received. All other reagents were reagent grade and purified where necessary. Reactions were monitored by thin layer chromatography (TLC) using EM Science 60F silica gel plates (0.25 mm). Compounds were visualized by dipping the plates in as cerium sulfate-ammonium molybdate solution, followed by heating. Flash column chromatography was performed over Scientific Adsorbents Inc. silica gel (32-63 mm). ¹H NMR and ¹³C NMR spectra were recorded on Bruker-Spectrospin spectrometers. The chemical shifts are reported as d values (ppm) relative to TMS. Coupling constants (J) are reported in hertz. Infrared spectra were recorded on a Perkin-Elmer Paragon 1000 FT-IR Spectrophotometer (NaCl plates, film). Low-Resolution mass spectra were performed on a JEOL LC/MS system using chemical ionization. High-resolution mass spectra were recorded on a JEOL-DX-303 HF mass spectrometer.

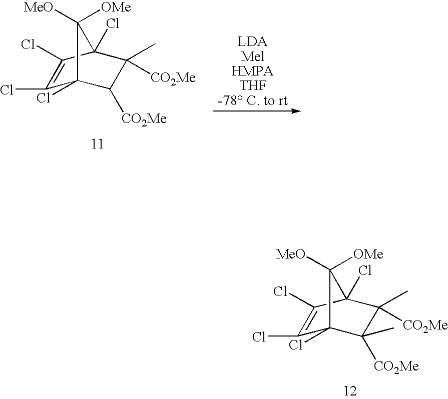

To a magnetically stirred solution of diisopropylamine (3.022 ml, 21.56 mmol) in anhydrous THF (80 mL) cooled to −78° C. was added dropwise n-BuLi (13.5 ml, 21.56 mmol) by syringe. After complete addition, the reaction mixture was warmed to 0° C. and stirred for 15 min. The reaction mixture was cooled to −78° C., to it was added dropwise a solution of the diester 11 (7.0 g, 16.59 mmol) in THF (40 mL), and stirring was continued at −78° C. for 1 hr and at −30° C. for another 1 hr. The mixture was again cooled to −78° C., and HMPA (4.996 mL, 28.72 mmol) was added followed by MeI (1.497 mL, 24.05 mmol). The reaction mixture was stirred at −78° C. for 1 hr and then slowly warmed up to rt and finally left at room temperature overnight. The reaction mixture, after quenched with saturated NH₄Cl, was extracted with CH₂Cl₂. The CH₂Cl₂ extract was dried over Na₂SO₄, filtered and concentrated in vacuo. Chromatography (0 to 10% EtOAc in Hexane) afforded 12 (6.83 g, 95%). ¹H NMR (CDCl₃, 400 MHz): δ 1.68 (s, 6H), 3.51 (s, 3H), 3.56 (s, 3H), 3.60 (s, 6H); ¹³C NMR (CDCl₃, 100 MHz): 18.8, 52.0, 52.5, 52.8, 61.1, 79.8, 113.2, 131.2, 171.5; IR (NaCl, cm⁻¹): 1750.3, 1727.2, 1254.0; HRMS Found: 434.9925 (M+H), Calc. for C₁₅H₁₉Cl₄O₆ 434.9857;

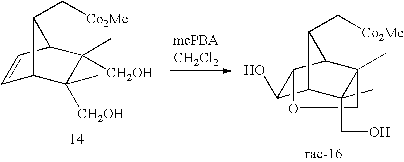

To a solution of diol 14 (980.3 mg, 3.85 mmol) in CH₂Cl₂ (25 mL) was added at 0° C. mCPBA (1722 mg, 7.71 mmol) in one portion. The reaction was slowly warmed up to room temperature and stirred overnight. The mixture was concentrated to reduce the volume to approximately 10 mL and then applied to SiO₂ column. Flash chromatography (50 to 100% EtOAc in hexanes) gave cyclic ether rac-16 (938.8 mg, 90%). ¹H NMR (CDCl₃, 400 MHz): δ 1.18 (s, 3H), 1.27 (s, 3H), 1.95 (s, 1H), 2.33 (d, J=5.1, 1H), 2.75-2.89 (m, 3H), 3.44 (d, J=9.0, 1H), 3.63 (d, J=9.2, 1H), 3.70 (s, 3H), 3.80 (d, J=9.0, 1H), 3.91 (s, 1H), 4.08 (d, J=5.3, 1H); ¹³C NMR (CDCl₃, 100 MHz): 18.8, 21.9, 34.4, 39.3, 44.5, 48.8, 51.8, 54.3, 55.4, 66.2, 76.7, 77.3, 88.4, 173.2; IR (NaCl, cm⁻¹): 3406.3, 2951.9, 2877.7, 1733.8, 1034.8; HRMS Found: 271.1538 (M⁺H), Calc. for C₁₄H₂₃O₅ 271.1467;

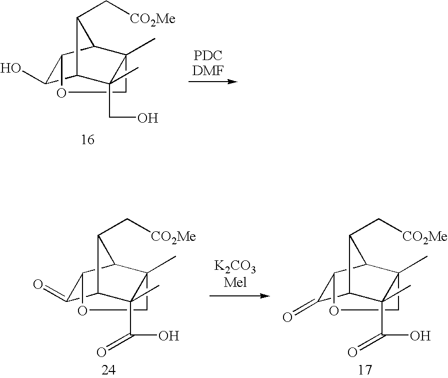

A solution of cyclic ether 16 (917.3 mg, 3.39 mmol) in DMF (18 mL) was treated with PDC (10.2 g, 27.15 mmol) at room temperature and stirred for 1 day. The reaction was worked up by pouring into water (100 mL) and thoroughly extracted with ether. The ether extraction was washed with brine, dried with MgSO₄, and concentrated in vacuo. The crude keto-acid 24 was dissolved in dry acetone (25 mL). Methyl iodide (20.1 mL, 33.9 mmol) and anhydrous potassium carbonate (4.7 g, 33.9 mmol) were added. After 10 hrs at reflux, the mixture was cooled, diluted with CH₂Cl₂, filtered and evaporated. The residue was dissolved in CH₂Cl₂ and purified by flash chromatography (20 to 50% EtOAc in Hexane) to afford keto-ester 17. ¹H NMR (C6D6, 400 MHz): δ 0.82 (s, 3H), 1.11 (s, 3H), 1.92 (t, J=8.0, 1H), 2.05 (d, J=5.6, 1H), 2.24 (d, J=8.0, 1H), 2.50 (s, 1H), 3.28 (s, 3H), 3.30 (s, 3H), 3.32 (d, J=8.9, 1H), 3.89 (d, J=5.6, 1H), 4.20 (d, J=8.9, 1H); ¹³C NMR (C6D6, 100 MHz) 17.7, 23.7, 34.2, 38.3, 51.6, 51.7, 51.8, 53.0, 54.9, 59.6, 79.6, 84.1, 171.1, 174.9, 203.2; IR (NaCl, cm⁻¹):1772.0, 1734.0; LRMS Found: 297.04 (M+H), Calc. C₁₅H₂₁O₆ 297.12.

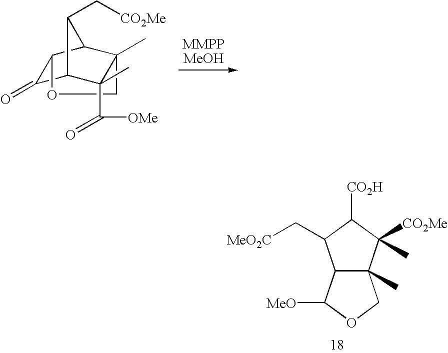

To a solution of keto-ester 17 (696.3 mg, 2.34 mmol) in MeOH (30 mL) was added MMPP (magnesium monoperoxyphthalate hexahydrate, tech 80%, 4.4 g, 7.02 mmol) in one portion at 0° C. After stirring at room temperature for 10 hrs, the white suspension was diluted with water, acidified with 1 M HCl to pH 2-3, and extracted 3 times with CH₂Cl₂. The organic extract was washed with brine, dried over Na₂SO₄, and rotary evaporated. Column chromatography (30 to 70% EtOAc in hexanes) gave carboxylic acid 18 (706.1 mg, 88%). ¹H NMR (CDCl₃, 400 MHz): δ 1.15 (s, 3H), 1.40 (s, 3H), 1.98 (dd, J=5.6, J=1.5, 1H), 2.44 (dd, J=15.8, J=9.7, 1H), 2.69 (d, J=10.3, 1H), 2.98 (dd, J=15.8, J=4.1, 1H), 3.17 (m, 1H), 3.30 (s, 3H), 3.48 (d, J=9.4, 1H), 3.67 (s, 3H), 3.68 (s, 3H), 4.04 (d, J=9.4, 1H), 4.73 (d, J=1.5, 1H); HRMS Found: 343.1383 (M−H), Calc. for C₁₆H₂₃O₈ 343.1471;

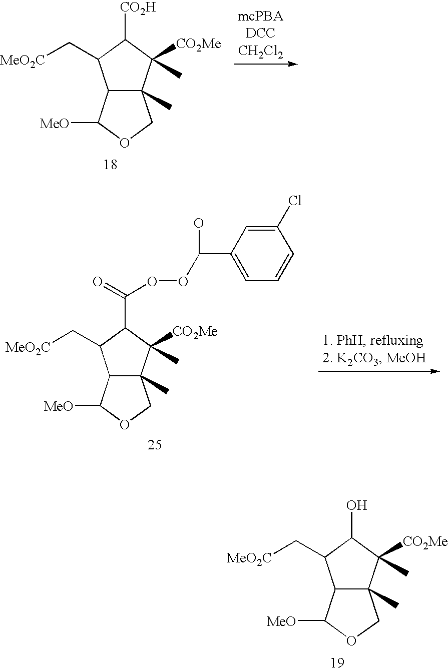

To a solution of carboxylic acid 17 (309.2 mg, 0.90 mmol) and mcPBA (401 mg, 1.80 mmol) in CH₂Cl₂ (10 mL) was added DCC (369.9 mg, 1.80 mmol.) at 0° C. with stirring. After 2 hr, the precipitate was filtered off. The filtrate was concentrated and subjected to flash chromatography (10 to 30% EtOAc in Hexanes) to give mixed peroxide 25 (369.2 mg, 83%). The mixed peroxide 25 (238.1 mg, 0.48 mmol) in benzene (15 mL) was refluxing for 10 hrs with stirring. The solvent was removed in vacuo. The residue was redissolved in dry MeOH (10 mL) and treated with anhydrous K₂CO₃ (263 mg, 1.91 mmol.). After stirring for 5 hrs at room temperature, the solution was diluted with CH2Cl2, filtered and evaporated. The residue was dissolved in CH₂Cl₂ and purified by flash chromatography (0 to 30% EtOAc in Hexanes) to afford 19 (105.5 mg, 70%). ¹H NMR (CDCl₃, 400 MHz): δ 1.14 (s, 3H), 1.26 (s, 3H), 1.85 (d, J=6.8, 1H), 2.39 (dd, J=15.6, J=9.4, 1H), 2.64 (m, 1H), 2.79 (dd, J=15.6, J=5.2, 1H), 3.06 (d, 11.1, 1H), 3.30 (s, 3H), 3.48 (d, J=9.4, 1H), 3.54 (dd, J=11.1, J=9.4, 1H), 3.69 (s, 3H), 3.72 (s, 3H), 4.12 (d, J=9.4, 1H), 4.78 (s, 1H); ¹³C NMR (CDCl₃, 75 MHz): 19.1, 21.5, 38.9, 45.1, 51.6, 52.0, 54.0, 54.8, 56.2, 61.4, 73.7, 84.6, 111.9, 172.9, 176.1; IR (NaCl, cm⁻¹): 3525.8, 2952.3, 1732.6, 1436.6; HRMS Found: 317.1597 (M+H), Calc. for C₁₅H₂₅O₇ 317.1522;

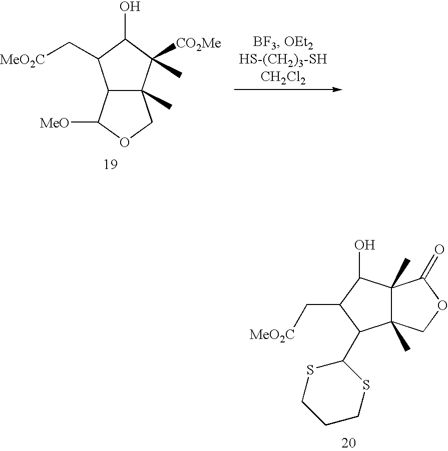

Boron trifluoride etherate (169 mL, 1.33 mmol.) was added dropwise to a solution of ketal 19 (105.5 mg, 0.33 mmol) and 1,3-propanedithol (201 mL, 2.00 nmol.) in CH₂Cl₂ (10 mL) at 0° C. The reaction was stirred at room temperature for 12 hrs, then poured into saturated NaHCO₃ and extracted 3 times with CH₂Cl₂. The CH₂Cl₂ extract was dried over Na₂SO₄, filtered and concentrated in vacuo. The residue was purified by flash chromatography to afford dithiane-lactone 20 (61.4 mg, 51%). ¹H NMR (CDCl₃, 400 MHz): δ 1.20 (s, 3H), 1.25 (s, 3H), 1.80-2.28 (m, 4H), 2.53 (dd, J=16.2, J=7.5, 1H), 2.82-2.95 (m, 5H), 3.43 (d, J=8.4, 1H), 3.66 (dd, J=17.3, J=8.7, 1H), 3.69 (s, 3H), 3.89 (d, J=10.0, 1H), 4.30 (d, J=4.4, 1H), 5.03 (d, J=10.0, 1H); HRMS Found: 361.1160 (M+H), Calc. For C₁₆H₂₅O₅S₂ 361.1065;

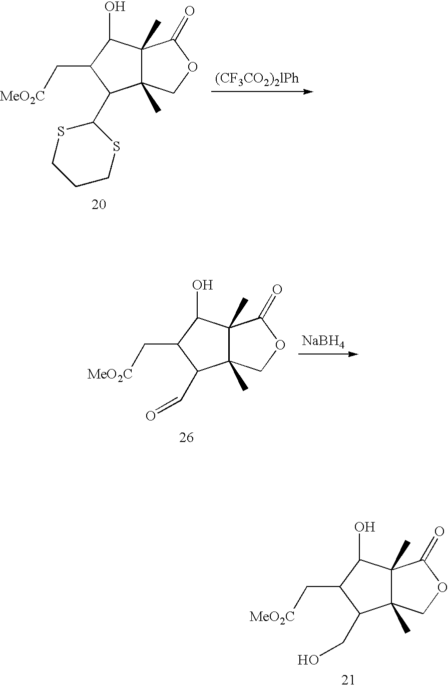

Bis(trifluoroacetoxy)iodobenzene (120 mg, 0.27 mmol.) was added at 0° C. to a stirred solution of dithiane-lactone 20 (61.0 mg, 0.17 mmol.), water (1 mL) and CH₃CN (9 mL). After it was stirred at room temperature for 10 min, the reaction was quenched with saturated sodium bicarbonate solution, and extracted 3 times with CH₂Cl₂. Drying (MgSO₄) and removal of solvents gave a residue which was purified by flash chromatography. (30 to 60% EtOAc in Hexanes) to give aldehyde 26 (23.0 mg, 50%). To a solution aldehyde 26 (23.0 mg, 0.085 mmol.) in MeOH (2 mL) was added at 0° C. NaBH₄ (6.5 mg, 0.17 mmol.). After the mixture was stirred at 0° C. for 1 h, HOAc (0.2 mL) was added. The mixture was then concentrated and the resulting residue was purified by flash chromatography (40 to 70% EtOAc in Hexanes) to yield diol 21 (23.4 mg, 100%). ¹H NMR (CDCl₃, 400 MHz): δ 1.11 (s, 3H), 1.29 (s, 3H), 1.44 (m, 1H), 1.93 (m, 1H), 2.34 (dd, J=16.9, J=8.0, 1H), 2.47 (dd, J=7.3, J=4.7, 1H), 2.81 (dd, J=16.9, J=4.1, 1H)), 3.44-3.80 (m, 4H), 3.70 (s, 3H), 3.82 (d, J=9.8, 1H)), 4.80 (d, J=9.8, 1H); ¹³C NMR (CDCl₃, 75 MHz): 15.6, 22.2, 29.7, 35.8, 42.5, 47.5, 52.1, 52.8, 59.9, 73.8, 77.2, 81.7, 174.3, 181.9; IR (NaCl, cm⁻¹): 3467.9, 2920.0, 1736.4; HRMS Found: 273.1337 (M+H), Calc. For C₁₃H₂₁O₆ 273.1260;

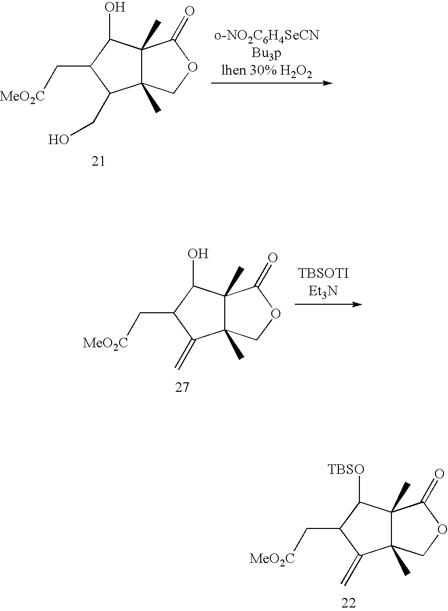

n-Tributylphosphine (46 mL, 0.18 mmol.) was added dropwise to a solution of diol 21 (10.1 mg, 0.037 mmol.) and onitrophenylselenocyanate (42 mg, 0.18 mmol.) in THF (2 mL). The whole solution quickly turned to red color. After stirring at room temperature for 2 hrs, the solution was concentrated and chromatographed (10 to 50% EtOAc in Hexanes) to give crude onitrophenyl selenide. Hydrogen peroxide (30%, 1 mL) was added to a solution of selenide in THF (2 mL) at 0° C. After stirring at room temperature overnight, the reaction mixture was poured into saturated Na2S2O3 and extracted 3 times with CH₂Cl₂. The organic layers were combined and dried over Na₂SO₄, filtered and oncentrated in vacuo. Residue was purified by column chromatography (0 to 30% EtOAc in Hexane) to give alcohol 27 (8.0 mg, 86%). To a solution of alcohol 27 (5.0 mg, 0.020 mmol) in CH₂Cl₂ (1 mL) was added Et₃N (8.2 mL, 0.060 mmol) then TBSOTf (9.0 mL, 0.040 mmol) at 0° C. The mixture was stirred at room temperature for 12 hrs. The reaction mixture, after quenched with 0.1N HCl, was extracted 3 times with CH₂Cl₂. The CH₂Cl₂ extract was dried over Na₂SO₄, filtered and concentrated in vacuo. Chromatography (0 to 10% EtOAc in Hexane) afforded 22 (5.6 mg, 76%). ¹H NMR (CDCl₃, 300 MHz): δ 0.07 (s, 3H), 0.11 (s, 3H), 0.88 (s, 9H), 1.18 (s, 3H), 1.19 (s, 3H), 2.48 (dd, J=15.8, J=7.3, 1H), 2.59 (dd, J=15.8, J=6.7, 1H), 3.05 (m, 1H), 3.71 (s, 3H), 3.89 (d, J=8.6, 1H), 3.90 (d, J=3.6, 1H), 4.19 (d, J=8.6, 1H), 4.99 (d, J=2.2, 1H), 5.04 (d, J=2.2, 1H); LRMS Found: 369.0 (M+1), Calc. 368.20.

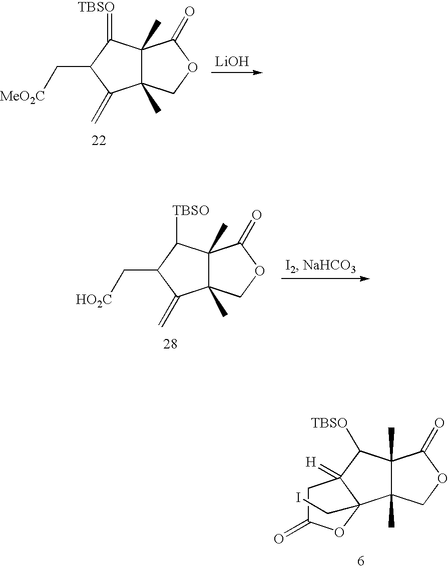

The ester 22 (4.0 mg, 0.011 mmol) was stirred with a solution of LiOH (1.4 mg, 0.033 mmol) in a mixture of MeOH (1.5 mL) and ater (0.5 mL) at room temperature for 12 hrs, diluted with water, acidified with 1 M HCl to pH 2-3, and extracted 3 times with CH₂Cl₂. The organic extract was washed with brine, dried over Na₂SO₄, and rotary evaporated. To a solution of crude carboxylic acid 28 in THF (0.5 mL), was added 1 mL of saturated aqueous NaHCO₃. The mixture was cooled in an ice bath, treated with a solution of I2 (8.2 mg, 0.033 mmol) in THF (1.5 mL), protected from light, and stirred at room temperature for 12 hrs. Excess I₂ was quenched by addition of saturated Na₂S₂O₃, the mixture was diluted with water and extracted 3 times with CH₂Cl₂. The organic extract was washed with brine, dried over Na₂SO₄, and rotary evaporated. Column chromatography (10 to 30% EtOAc in hexanes) gave iodolactone 6 (4.0 mg, 75%). ¹H NMR (CDCl₃, 400 MHz): δ 0.074 (s, 3H), 0.077 (s, 3H), 0.88 (s, 9H), 1.16 (s, 3H), 1.23 (s, 3H), 2.45 (dd, J=19.1, J=2.3, 1H), 2.79 (dd, J=11.5, J=2.3, 1H), 3.34 (d, J=11.1, 1H), 3.35 (dd, J=19.1, J=11.5, 1H), 3.56 (d, J=11.1, 1H), 3.82 (s, 1H), 3.88 (d, J=8.4, 1H), 4.30 (d, J=8.4, 1H); ¹³C NMR (CDCl₃, 100 MHz): −5.0, −4.6, 8.0, 16.0, 16.4, 17.9, 25.7, 37.5, 56.1, 57.2, 61.3, 72.4, 87.9, 95.5, 173.7, 175.9; HRMS Found: 481.0907 (M+H), Calc. For C₁₈H₃₁O₅SiI 481.0829;

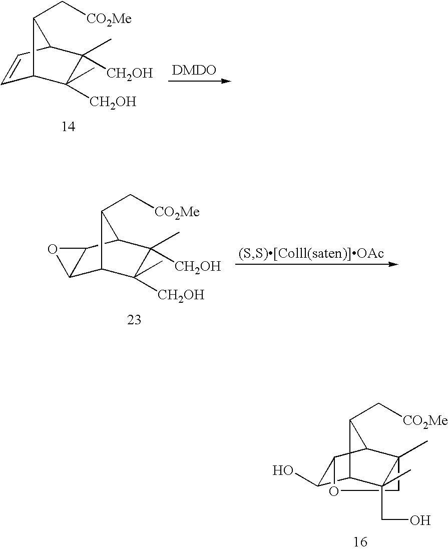

To a solution of diol 14 (21 mg, 0.083 mmol) in CH₂Cl₂ (1 mL) was added a solution of DMDO in acetone (˜0.07 M, 3.5 mL) at room temperature. The reaction mixture was then stirred for 20 min. The solvent was removed to afford the crude epoxide 23. ¹H NMR (CDCl₃, 300 MHz): δ 1.30 (s, 6H), 2.22 (s, 2H), 2.38 (t, J=8.8, 1H), 2.83 (d, J=8.8, 2H), 3.08 (br, 2H), 3.37 (s, 2H), 3.50 (d, J=10.9, 2H), 3.69 (s, 3H), 4.21, (d, J=10.9, 2H). The crude epoxide was dissolved in THF (0.5 mL) and cooled to −78° C. To this solution was added (S,S)-[Co^{III}(salen)]-OAc (16 mg, 025 mmol, 0.3 eq.). The mixture was stirred at −78° C. for 48 hr and kept in −25° C. freezer for 48 hr. The reaction mixture was loaded directly onto a SiO₂ column and purified by flash chromatography (50 to 100% EtOAc in Hexane) to afford of asymmetric 16 (19 mg, 86%). The enantiomers were analyzed by chiral HPLC as benzyl ester using a Chiracel AD column (15% IPA in hexanes, 1 ml/min, tR=15.41, 18.33 min).

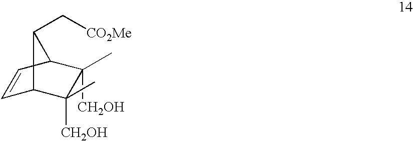

Magnesium turnings (20 eq., 138 mmol, 3.35 g) were added to a solution of unsaturated ester 13 (2108 MG, 6.88 mmol) in MeOH (60 mL) at 0° C. portionwise. The reaction was slowly warmed up to room temperature and stirred overnight. (caution: A large amount of gas is generated.) The mixture was cooled to 0° C., THF (40 mL) and then 3N HCl were added till pH=1. The mixture was stirred for 1 h and then thoroughly extracted with CH₂Cl₂. The organic layer was dried over Na₂SO₄, concentrated in vacuo and purified to give diol 14 (1342 mg, 77%) ¹H NMR (CDCl₃, 400 MHz): δ 1.34 (s, 6H), 2.15 (6, J=8.5, 1H), 2.38 (d, J=1.2, 1H), 2.81 (d, J=8.5, 1H), 3.02 (brs, 2H), 3.28 (d, J=11.2, 1H), 3.56 (d, J=11.2, 1H), 3.68 (s, 3H), 6.25 (t, J=2.0, 1H); ¹³C NMR (CDCl₃, 100 MHz): 22.0, 37.2, 49.3, 51.7, 57.0, 57.9, 71.7, 137.4, 172.8; IR (NaCl, cm⁻¹): 3221.8, 2952.3, 2876.2, 1737.6, 1034.1; HRMS Found: 255.1604 (M+H), Calc. for C₁₄H₂₂O₄ 254.15;

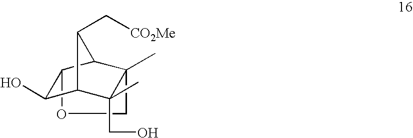

To a solution of diol 14 (980.3 mg, 3.85 mmol) in CH₂Cl₂ (25 mL) was added at 0° C. mCPBA (1722 mg, 7.71 mmol) in one portion. The reaction was slowly warmed up to room temperature and stirred overnight. The mixture was concentrated to reduce the volume to approximately 10 mL and then applied to SiO₂ column. Flash chromatography (50 to 100% EtOAc in hexanes) gave cyclic ether rac-16 (938.8 mg, 90%). ¹H NMR (CDCl₃, 400 MHz): δ 1.18 (s, 3H), 1.27 (s, 3H), 1.95 (s, 1H), 2.33 (d, J=5.1, 1H), 2.75-2.89 (m, 3H), 3.44 (d, J=9.0, 1H), 3.63 (d, J=9.2, 1H), 3.70 (s, 3H), 3.80 (d, J=9.0, 1H), 3.91 (s, 1H), 4.08 (d, J=5.3, 1H); ¹³C NMR (CDCl₃, 100 MHz): 18.8, 21.9, 34.4, 39.3, 44.5, 48.8, 51.8, 54.3, 55.4, 66.2, 76.7, 77.3, 88.4, 173.2; IR (NaCl, cm⁻¹): 3406.3, 2951.9, 2877.7, 1733.8, 1034.8; HRMS Found: 271.1538 (M+H), Calc. for C₁₄H₂₃O₅ 271.1467;

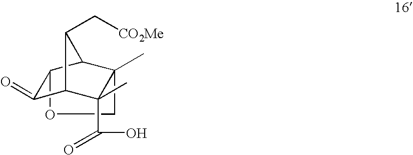

A solution of cyclic ether 16 (917.3 mg, 3.39 mmol) in DMF (18 mL) was treated with PDC (10.2 g, 27.15 mmol) at room temperature and stirred for 1 day. The reaction was worked up by poured into water (100 mL) and thoroughly extracted with ether. The ether extraction was washed with brine, dried with MgSO₄, and concentrated in vacuo to afford 16′.

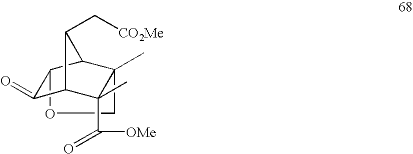

The crude keto-acid 16′ was dissolved in dry acetone (25 mL). Methyl iodide (20.1 mL, 33.9 mmol) and anhydrous potassium carbonate (4.7 g, 33.9 mmol) were added. After 10 hrs at reflux, the mixture was cooled, diluted with CH₂Cl₂, filtered and evaporated. The residue was dissolved in CH₂Cl₂ and purified by flash chromatography (20 to 50% EtOAc in Hexane) to afford keto-ester 68. ¹H NMR (C₆D₆, 400 MHz): 80.82 (s, 3H), 1.11 (s, 3H), 1.92 (t, J=8.0, 1H), 2.05 (d, J=5.6, 1H), 2.24 (d, J=8.0, 1H), 2.50 (s, 1H), 3.28 (s, 3H), 3.30 (s, 3H), 3.32 (d, J=8.9, 1H), 3.89 (d, J=5.6, 1H), 4.20 (d, J=8.9, 1H); ¹³C NMR (C₆D₆, 100 MHz): 17.7, 23.7, 34.2, 38.3, 51.6, 51.7, 51.8, 53.0, 54.9, 59.6, 79.6, 84.1, 171.1, 174.9, 203.2; IR (NaCl, cm¹): 1772.0, 1734.0; HRMS Found: 297.1334 (M+H), Calc. C₁₅H₂₀O₆ 296.13.

(R,R)-68 [α]D²¹=51.8 (c=0.11, CHCl₃)

(S,S)-68 [α]D²¹=−45.1 (c=0.43, CHCl₃)

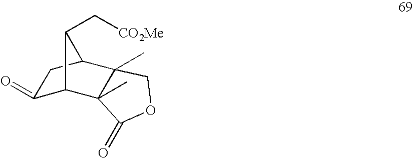

SmI₂ (0.1M conc., 1.2 mL, 1.2 mmol) was added to a degassed solution of keto ester 68 (0.034 mmol) in THF (5 mL) and MeOH (2 mL) at −78° C. After stirring at −78° C. for 0.5 h, the mixture was quenched with careful addition of sat. Na₂S₂O₃ solution (1 mL). The mixture was warmed up to room temperature and extracted with CH₂Cl₂. The organic layer was dried over Na₂SO₄, concentrated. Column chromatography (30-60% EtOAc/Hexane) afforded ketolactone 69 (6.5 mg, 72%). ¹H NMR (CDCl₃, 400 MHz): δ 1.26 (s, 3H), 1.35 (s, 3H), 2.21 (d, J=18.7, 1H), 2.30 (dd, J=18.7, J=4.4, 1H), 2.34 (d, J=4.4, 1H), 2.53 (d, J=8.2, 1H), 2.59 (m, 1H), 2.67 (s, 1H), 2.80 (d, J=8.2, 1H), 3.73 (s, 3H), 3.76 (d, J=10.2, 1H), 4.27 (d, J=10.2, 1H).

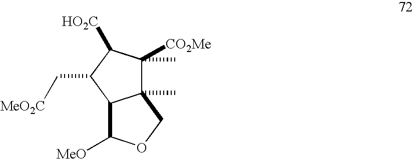

To a solution of keto-ester 68 (696.3 mg, 2.34 mmol) in MeOH (30 mL) was added MMPP (magnesium monoperoxyphthalate hexahydrate, tech 80%, 4.4 g, 7.02 mmol) in one portion at 0° C. After stirring at room temperature for 10 hrs, the white suspension was diluted with water, extracted 3 times with CH₂Cl₂. The organic extract was washed with brine, dried over Na₂SO₄, and rotary evaporated. Column chromatography (30 to 70% EtOAc in hexanes) gave carboxylic acid 72 (706.1 mg, 88%). ¹H NMR (CDCl₃, 400 MHz): δ 1.15 (s, 3H), 1.40 (s, 3H), 1.98 (dd, J=5.6, J=1.5, 1H), 2.44 (dd, J=15.8, J=9.7, 1H), 2.69 (d, J=10.3, 1H), 2.98 (dd, J=15.8, J=4.1, 1H), 3.17 (m, 1H), 3.30 (s, 3H), 3.48 (d, J=9.4, 1H), 3.67 (s, 3H), 3.68 (s, 3H), 4.04 (d, J=9.4, 1H), 4.73 (d, J=1.5, 1H); HRMS Found: 343.1383 (M−H), Calc. for C₁₆H₂₃O₈ 343.1471;

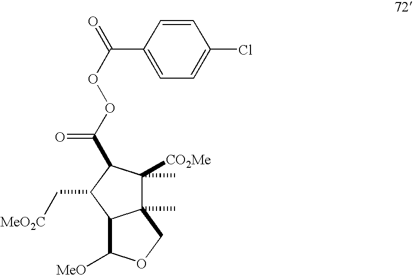

To a solution of carboxylic acid 72 (309.2 mg, 0.90 mmol) and mcPBA (401 mg, 1.80 mmol) in CH₂Cl₂ (10 mL) was added DCC (369.9 mg, 1.80 mmol.) at 0° C. with stirring. After 2 hr, the precipitate was filtered off. The filtrate was concentrated and subjected to flash chromatography (10 to 30% EtOAc in Hexanes) to give mixed peroxide 72′ (369.2 mg, 83%). ¹H NMR (CDCl₃, 400 MHz): δ 1.20 (s, 3H), 1.47 (s, 3H), 2.06 (dd, J=5.8, J=1.3, 1H), 2.50 (dd, J=16.0, J=9.4, 1H), 2.94 (dd, J=16, J=4.2, 1H), 2.95 (d, J=16, 1H), 2.79 (dd, J=15.6, J=5.2, 1H), 3.06 (d, 11.1, 1H), 3.30 (s, 3H), 3.48 (d, J=9.4, 1H), 3.30 (s, 3H), 3.33 (m, 1H), 3.50 (d, J=9.5, 1H), 3.69 (s, 3H), 3.72 (s, 3H), 4.08 (d, J=9.5, 1H), 4.77 (d, J=1.3, 1H), 7.41-7.98 (m, 4H); ¹³C NMR (CDCl₃, 75 MHz): 21.0, 21.2, 39.9, 40.9, 51.7, 52.5, 55.1, 55.7, 57.4, 58.7, 63.9, 72.6, 112.3, 126.9, 127.5, 129.4, 129.9, 134.1, 134.7, 161.3, 167.7, 171.8, 174.2; IR (NaCl, cm⁻¹): 2952.6, 1800.3, 1771.0, 1732.7, 1435.3, 1225.0, 1099.9; HRMS Found: 497.1241 (M−H), Calc. for C₂₃H₂₇ClO₁₀ 498.13;

(R,R)-72′ (α)D²¹=−61.2 (c=0.26, CHCl₃)

(S,S)-72′ [α]D²¹=62.3 (c=0.38, CHCl₃)

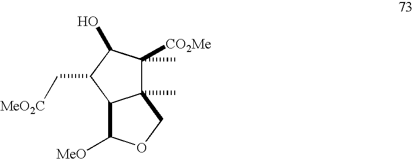

The mixed peroxide 72′ (238.1 mg, 0.48 mmol) in benzene (15 mL) was refluxing for 10 hrs with stirring. The solvent was removed in vacuo. The residue was redissolved in dry MeOH (10 mL) and treated with anhydrous K₂CO₃ (263 mg, 1.91 mmol.). After stirring for 5 hrs at room temperature, the solution was diluted with CH₂Cl₂, filtered and evaporated. The residue was dissolved in CH₂Cl₂ and purified by flash chromatography (0 to 30% EtOAc in Hexanes) to afford 73 (105.5 mg, 70%). ¹H NMR (CDCl₃, 400 MHz): δ 1.14 (s, 3H), 1.26 (s, 3H), 1.85 (d, J=6.8, 1H), 2.39 (dd, J=15.6, J=9.4, 1H), 2.64 (m, 1H), 2.79 (dd, J=15.6, J=5.2, 1H), 3.06 (d, 11.1, 1H), 3.30 (s, 3H), 3.48 (d, J=9.4, 1H), 3.54 (dd, J=11.1, J=9.4, 1H), 3.69 (s, 3H), 3.72 (s, 3H), 4.12 (d, J=9.4, 1H), 4.78 (s, 1H); ¹³C NMR (CDCl₃, 75 MHz): 19.1, 21.5, 38.9, 45.1, 51.6, 52.0, 54.0, 54.8, 56.2, 61.4, 73.7, 84.6, 111.9, 172.9, 176.1; IR (NaCl, cm⁻¹): 3525.8, 2952.3, 1732.6, 1436.6; HRMS Found: 317.1597 (M+H), Calc. for C₁₅H₂₅O₇ 317.1522;

(R,R)-73 [α]D²¹=−81.0 (c=0.18, CHCl₃)

(s,S)-73 [α]D²¹=70.6 (c=0.68, CHCl₃)

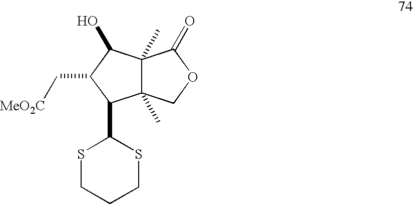

Boron trifluoride etherate (169 μL, 1.33 mmoL) was added dropwise to a solution of ketal 73 (105.5 mg, 0.33 mmol) and 1,3-propanedithol (201 μL, 2.00 mmol.) in CH₂Cl₂ (10 mL) at 0° C. The reaction was stirred at room temperature for 12 hrs, then poured into saturated NaHCO₃ and extracted 3 times with CH₂Cl₂. The CH₂Cl₂ extract was dried over Na₂SO₄, filtered and concentrated in vacuo. The residue was purified by flash chromatography to afford dithiane-lactone 74 (61.4 mg, 51%). ¹H NMR (CDCl₃, 400 MHz): δ 1.20 (s, 3H), 1.25 (s, 3H), 1.80-2.28 (m, 4H), 2.53 (dd, J=16.2, J=7.5, 1H), 2.82-2.95 (m, 5H), 3.43 (d, J=8.4, 1H), 3.66 (dd, J=17.3, J=8.7, 1H), 3.69 (s, 3H), 3.89 (d, J=10.0, 1H), 4.30 (d, J=4.4, 1H), 5.03 (d, J=10.0, 1H); HRMS Found: 361.1160 (M+H), Calc. For C₁₆H₂₅O₅S₂ 361.1065;

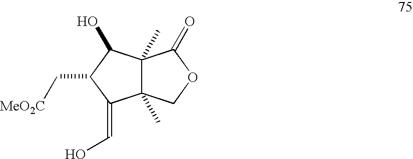

Bis(trifluoroacetoxy)iodobenzene (120 mg, 0.27 mmol.) was added at 0° C. to a stirred solution of dithiane-lactone 74 (61.0 mg, 0.17 mmol.), water (1 mL) and CH₃CN (9 mL). After it was stirred at room temperature for 10 min, the reaction was quenched with saturated sodium bicarbonate solution, and extracted 3 times with CH₂Cl₂. Drying (MgSO₄) and removal of solvents gave a residue which was purified by flash chromatography (30 to 60% EtOAc in Hexanes) to give aldehyde 74′ (23.0 mg, 50%). To a solution aldehyde 74′ (23.0 mg, 0.085 mmol.) in MeOH (2 mL) was added at 0° C. NaBH₄ (6.5 mg, 0.17 mmol.). After the mixture was stirred at 0° C. for 1 h, HOAC (0.2 mL) was added. The mixture was then concentrated and the resulting residue was purified by flash chromatography (40 to 70% EtOAc in Hexanes) to yield diol 75 (23.4 mg, 100%). ¹H NMR (CDCl₃, 400 MHz): δ 1.11 (s, 3H), 1.29 (s, 3H), 1.44 (m, 1H), 1.93 (m, 1H), 2.34 (dd, J=16.9, J=8.0, 1H), 2.47 (dd, J=7.3, J=4.7, 1H), 2.81 (dd, J=16.9, J=4.1, 1H)), 3.44-3.80 (m, 4H), 3.70 (s, 3H), 3.82 (d, J=9.8, 1H)), 4.80 (d, J=9.8, 1H); ¹³C NMR (CDCl₃, 75 MHz): 15.6, 22.2, 29.7, 35.8, 42.5, 47.5, 52.1, 52.8, 59.9, 73.8, 77.2, 81.7, 174.3, 181.9; IR (NaCl, cm⁻¹): 3467.9, 2920.0, 1736.4; HRMS Found: 273.1337 (M+H), Calc. For C₁₃H₂₁O₆ 273.1260;

(R,R)-75 [α]D²=30.3 (c=0.22, CHCl₃)

(S,S)-75 [α]D²¹=−28.5 (c=0.59, CHCl₃)

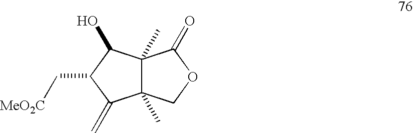

n-Tributylphosphine (46 μL, 0.18 mmol.) was added dropwise to a solution of diol 75 (10.1 mg, 0.037 mmol.) and o-nitrophenylselenocyanate (42 mg, 0.18 mmol.) in THF (2 mL). The whole solution quickly turned to red color. After stirring at room temperature for 2 hrs, the solution was concentrated and chromatographed (10 to 50% EtOAc in Hexanes) to give crude o-nitrophenyl selenide. Hydrogen peroxide (30%, 1 mL) was added to a solution of selenide in THF (2 mL) at 0° C. After stirring at room temperature overnight, the reaction mixture was poured into saturated Na₂S₂O₃ and extracted 3 times with CH₂Cl₂. The organic layers were combined and dried over Na₂SO₄, filtered and concentrated in vacuo. Residue was purified by column chromatography (0 to 30% EtOAc in Hexane) to give alcohol 76 (8.0 mg, 86%). ¹H NMR (CDCl₃, 400 MHz): δ 1.14 (s, 3H), 1.32 (s, 3H), 2.62 (m, 3H), 2.39 (dd, J=15.6, J=9.4, 1H), 2.64 (m, 1H), 3.47 (d, J=10.7, 1H), 3.67 (m, 1H), 3.69 (s, 3H), 4.06 (d, J=8.8, 1H), 4.27 (d, J=8.8, 1H), 5.01 (d, J=1.6, 1H), 5.15 (d, J=1.6, 1H); ¹³C NMR (CDCl₃, 100 MHz): 15.1, 19.6, 35.0, 47.0, 50.3, 51.8, 52.0, 78.7, 80.6, 108.9, 155.3, 172.1, 181.4; IR (NaCl, cm⁻¹): 3505.0, 2970.0, 1740.1, 1436.6; LRMS Found: 255.11 (M+H), Calc. for C₁₃H₁₈O₅ 254.12;

(R,R)-76 [α]_{D}²¹=62.6 (c=0.68, CHCl₃)

(S,S)-76 [α]_{D}²¹=−63.1 (c=0.85, CHCl₃)

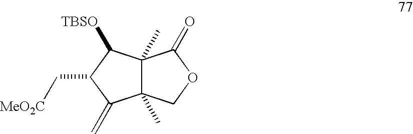

To a solution of alcohol 76 (5.0 mg, 0.020 mmol) in CH₂Cl₂ (1 mL) was added Et₃N (8.2 μL, 0.060 mmol) then TBSOTf (9.0 μL, 0.040 mmol) at 0° C. The mixture was stirred at room temperature for 12 hrs. The reaction mixture, after quenched with 0.1N HCl, was extracted 3 times with CH₂Cl₂. The CH₂Cl₂ extract was dried over Na₂SO₄, filtered and concentrated in vacuo. Chromatography (0 to 10% EtOAc in Hexane) afforded 77 (5.6 mg, 76%). ¹H NMR (CDCl₃, 300 MHz): δ 0.07 (s, 3H), 0.11 (s, 3H), 0.88 (s, 9H), 1.18 (s, 3H), 1.19 (s, 3H), 2.48 (dd, J=15.8, J=7.3, 1H), 2.59 (dd, J=15.8, J=6.7, 1H), 3.05 (m, 1H), 3.71 (s, 3H), 3.89 (d, J=8.6, 1H), 3.90 (d, J=3.6, 1H), 4.19 (d, J=8.6, 1H), 4.99 (d, J=2.2, 1H), 5.04 (d, J=2.2, 1H); ¹³C NMR (CDCl₃, 100 MHz): −4.9, −4.5, 16.4, 17.7, 20.0, 25.6, 37.6, 49.7, 51.7, 53.4, 57.8, 83.6, 108.6, 156.7, 172.0, 177.9; IR (NaCl, cm⁻¹): 2954.0, 1772.1.1, 1738.1.9, 1249.1; LRMS Found: 369.22 (M+1), Calc. 368.20;

(R,R)-77 [α]D²¹=16.6 (c=0.36, CHCl₃)

(S,S)-77 [α]D²¹=−15.3 (c=0.73, CHCl₃)

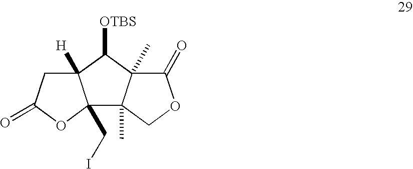

The ester 77 (4.0 mg, 0.011 mmol) was stirred with a solution of LiOH (1.4 mg, 0.033 mmol) in a mixture of MeOH (1.5 mL) and water (0.5 mL) at room temperature for 12 hrs, diluted with water, acidified with 1 M HCl to pH 2-3, and extracted 3 times with CH₂Cl₂. The organic extract was washed with brine, dried over Na₂SO₄, and rotary evaporated. To a solution of crude carboxylic acid 77′ in THF (0.5 mL), was added 1 mL of saturated aqueous NaHCO₃. The mixture was cooled in an ice bath, treated with a solution of 12 (8.2 mg, 0.033 mmol) in THF (1.5 mL), protected from light, and stirred at room teperature for 12 hrs. Excess 12 was quenched by addition of saturated Na₂S₂O₃, the mixture was diluted with water and extracted 3 times with CH₂Cl₂. The organic extract was washed with brine, dried over Na₂SO₄, and rotary evaporated. Column chromatography (10 to 30% EtOAc in hexanes) gave iodolactone 29 (4.0 mg, 75%). ¹H NMR (CDCl₃, 400 MHz): δ 0.074 (s, 3H), 0.077 (s, 3H), 0.88 (s, 9H), 1.16 (s, 3H), 1.23 (s, 3H), 2.45 (dd, J=19.1, J=2.3, 1H), 2.79 (dd, J=11.5, J=2.3, 1H), 3.34 (d, J=11.1, 1H), 3.35 (dd, J=19.1, J=11.5, 1H), 3.56 (d, J=11.1, 1H), 3.82 (s, 1H), 3.88 (d, J=8.4, 1H), 4.30 (d, J=8.4, 1H); ¹³C NMR (CDCl₃, 100 MHz): −5.0, −4.6, 8.0, 16.0, 16.4, 17.9, 25.7, 37.5, 56.1, 57.2, 61.3, 72.4, 87.9, 95.5, 173.7, 175.9; IR (NaCl, cm⁻¹): 2931.0, 1786.1, 1769.9; HRMS Found: 481.0907 (M+H), Calc. For C₁₈H₃₀₁O₅SiI 481.0829;

(R,R)-13 [α]D²¹=−6.1 (c=0.28, CHCl₃)

(S,S)-13 [α]D²¹=5.4 (c=0.48, CHCl₃)

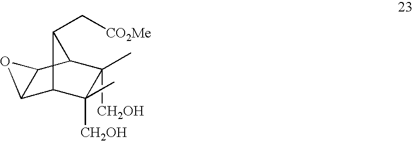

To a solution of diol 14 (21 mg, 0.083 mmol) in CH₂Cl₂ (1 mL) was added a solution of DMDO in acetone (˜0.07 M, 3.5 mL) at room temperature. The reaction mixture was then stirred for 20 min. The solvent was removed to afford the crude epoxide 23. ¹H NMR (CDCl₃, 300 MHz): δ 1.30 (s, 6H), 2.22 (s, 2H), 2.38 (t, J=8.8, 1H), 2.83 (d, J=8.8, 2H), 3.08 (br, 2H), 3.37 (s, 2H), 3.50 (d, J=10.9, 2H), 3.69 (s, 3H), 4.21, (d, J=10.9, 2H). The crude epoxide was dissolved in THF (0.5 mL) and cooled to −78° C. To this solution was added (S,S)-[Co^{III}(salen)]-OAc (16 mg, 0.025 mmol, 0.3 eq.). The mixture was stirred at −78° C. for 48 hr and kept in −25° C. freezer for 48 hr. The reaction mixture was loaded directly onto a SiO₂ column and purified by flash chromatography (50 to 100% EtOAc in Hexane) to afford of asymmetric 16 (19 mg, 86%). The enantiomers were analyzed by chiral HPLC as benzyl ester using a Chiracel AD column (15% IPA in hexanes, 1 ml/min, t_{R}=15.41, 18.33 min).

(R,R)-90 [α]D²¹=7.9 (c=0.46, CHCl₃)

(S,S)-90 [α]D²¹=−10.9 (c=0.2, CHCl₃)

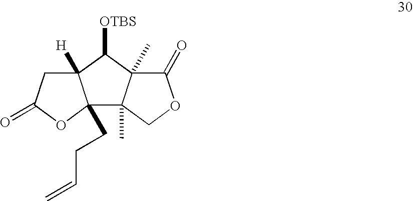

Iodolactone 29 (431 mg, 0.898 mmol), allyltributyltin (1.36 mL, 4.39 mmol), AIBN (28 mg, 0.17 mmol), and benzene (6 mL) were added into a flask equipped with a reflux condenser and a magnetic stirring bar, the mixture was degassed using the freeze-pump-thaw technique (3-4 cycles) and immersed into an oil bath kept at 85° C. After 3 hours, the mixture was cooled, the solvent was rotary evaporated, and the residue was chromatographed (10% KF in SiO₂, hexanes/EtOAc 7:1) to afford 262 mg (74% yield). ¹H NMR (CDCl₃, 500 MHz): δ 0.06 (s, 3H), 0.07 (s, 3H), 0.88 (s, 9H), 1.17 (s, 3H), 1.23 (s, 3H), 1.56 (m, 1H), 1.98 (m, 1H), 2.05 (m, 1H), 2.17 (m, 1H), 2.54 (dd, J=8.8, J=1.5, 1H), 2.71 (d, J=10.9, 1H), 3.00 (dd, J=18.8, J=10.9, 1H), 3.78 (s, 1H), 3.87 (d, J=8.6, 1H), 4.21 (d, J=8.6, 1H), 5.05 (d, J=10.2, 1H), 5.10 (dd, J=17.2, J=1.2, 1H), 5.77 (d, J=10.3, 1H), 5.80 (m, 1H); ¹³C NMR (CDCl₃, 100 MHz): −5.2, −4.8, 16.2, 16.4, 17.8, 25.6, 27.7, 33.9, 36.5, 54.0, 58.0, 60.3, 72.5, 89.0, 98.3, 116.1, 136.5, 174.6, 176.6; IR (NaCl, cm⁻¹): 1779 s (C═O); MS Found: 395.2 (M+1), Calc. 394.22;

(R,R)-87 [α]D²¹=8.9 (c=0.32, CHCl₃)

(S,S)-87 [α]D²¹=−9.3 (c=0.67, CHCl₃)

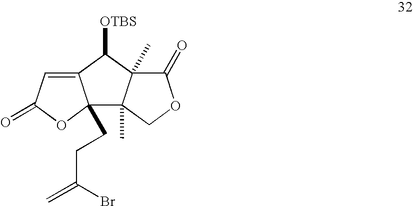

To a solution of 30 (262 mg, 0.665 mmol) in 18 mL of THF stirring at −78° C. was added LHMDS (1 M in THF, 3 eq. 2.0 mL). After 1.5 hour, PhSeCl (191 mg, 0.998 mmol, 1.5 eq.) in 3 mL of THF was added quickly. The mixture was allowed to warm to room temperature over 1.5 hours, diluted with water, and extracted with CH₂Cl₂ 3 times. The extract was dried over Mg304, rotary evaporated. The crude selenide was dissolved in 7 mL of dry MeCN and treated with a solution of PhSeBr (˜1.2 eq., 0.798 mmol, 188 mg) until brownish color persisted at RT. After 0.5 hour, the mixture was evaporated at 25° C. by rotavap, the residue redissolved in 20 ml of CH₂Cl₂, and ozonated at −78° C. until blue color persisted. The cold mixture was treated with 3 mL of 1-hexene and then added in several portions to a boiling solution of 2 mL of NEt₃ in 80 mL of benzene. After the addition was complete, the mixture was refluxed for 0.5 hour, evaporated to dryness, and the residue was chromatographed (hexanes/EtOAc 4:1) to afford 32 (175 mg, 56% yield). ¹H NMR (CDCl₃, 400 MHz): 80.17 (s, 3H), 0.19 (s, 3H), 0.90 (s, 9H), 0.91 (s, 3H), 1.20 (s, 3H), 2.18-2.26 (m, 1H), 2.32-2.49 (m, 3H), 3.93 (d, J=10.2, 1H), 4.36 (9, 1H), 4.68 (d, J=10.2, 1H), 5.42 (d, J=2.0, 1H), 5.57 (dd, J=1.0, J=0.8, 1H), 5.93 (s, 1H); ¹³C NMR (CDCl₃, 100 MHz): −4.8, −4.7, 16.3, 18.6, 25.9, 32.5, 35.6, 49.7, 59.7, 71.7, 73.9, 94.6, 114.3, 117.4, 131.8, 171.0, 171.6, 175.3; IR (NaCl, cm⁻¹): 1766 s (C═O); MS Found: 471.0 (M+1), Calc. 470.11;

(R,R)-14 [α]D²¹=−7.9 (c=0.46, CHCl₃)

(S,S)-14 [α]D²¹=8.6 (c=0.2, CHCl₃)

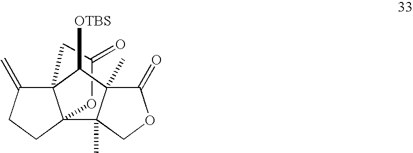

A solution of 32 (175 mg, 0.372 mmol), Bu₃SnH (200 μL, 0.727 mmol), and AIBN (16 mg, 0.098 mmol) in 50 mL of benzene was degassed using the freeze-pump-thaw technique (3 cycles) and heated under reflux in an oil bath at 85° C. After 5 hrs, the mixture was evaporated, and the residue was chromatographed (10% KF in SiO₂, hexanes/EtOAc 7:1) to afford 125 mg of 33 (86% yield). ¹H NMR (CDCl₃, 400 MHz): δ 0.01 (s, 3H), 0.06 (s, 3H), 0.86 (s, 9H), 1.21 (s, 3H), 1.23 (s, 3H), 1.76 (m, 1H), 2.11 (m, 1H), 2.61 (m, 2H), 2.79 (d, J=19.2, 1H), 3.03 (d, J=19.2, 1H), 3.89 (d, J=8.4, 1H), 4.00 (s, 1H), 4.43 (d, J=8.4, 1H), 4.95 (app s, 1H), 5.25 (dd, J=1.9, J=1.7, 1H); ¹³C NMR (CDCl₃, 100 MHz): −4.1, −3.1, 16.8, 17.9, 18.0, 26.0, 33.9, 37.7, 43.6, 56.9, 62.5, 66.3, 72.4, 89.1, 106.2, 112.1, 152.6, 174.2, 176.7; IR (NaCl, cm⁻¹): 1778 s (C═O); MS Found: 393.16 (M+1), Calc. 392.20;

(R,R)-15 [α]D²¹=−7.7 (c=0.36, CHCl₃)

(S,S)-15 [α]D²¹=7.9 (c=0.61, CHCl₃)

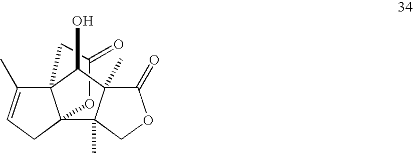

A mixture of 33 (125 mg, 0.318 mmol), p-TsOH.H₂O (242 mg, 1.27 mmol), and benzene (17 mL) was heated under reflux for 3 hours in an oil bath at 90° C., then cooled, diluted with Et₂O, and washed with aqueous NaHCO₃. The aqueous wash was extracted with CH₂Cl₂ 3 times, the combined organic phase was dried over Na₂SO₄, rotary evaporated, and chromatographed (CH₂Cl₂/EtOAc 5:1) to afford 89 mg (100%) of the product. ¹H NMR (CD₃OD, 400 MHz): δ 1.15 (s, 3H), 1.19 (d, J=0.8, 3H), 1.79 (ddd, J=2.4, J=2.1, J=1.5, 3H), 2.35 (ddq, J=18.4, J=2.4, J=2.4, 1H), 2.55 (ddq, J=18.4, J=2.1, J=2.1, 1H), 2.77 (d, J=19.3, 1H), 2.87 (d, J=19.3, 1H), 3.97 (d, J=8.6, 1H), 4.08 (s, 1H), 4.16 (d, J=8.6, J=0.8, 1H), 5.33 (ddq, J=2.4, J=2.1, J=1.5, 1H); ¹³C NMR (CD₃OD, 100 MHz): δ 15.2, 16.2, 17.0, 40.6, 41.9, 57.0, 64.0, 71.5, 74.3, 86.8, 106.3, 124.8, 143.4, 177.4, 179.7; IR (NaCl, cm⁻¹): 1766 s (C═O); MS Found: 279.19 (M+1), Calc. 278.12;

(R,R)-3 [α]D²¹=53.3 (c=0.21, MeOH)

(S,S)-3 [α]D²¹=−48.1 (c=0.65, MeOH)

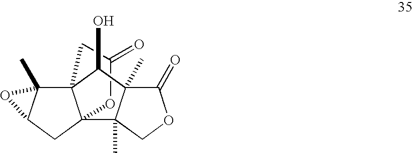

A solution of alcohol 34 (89 mg, 0.302 mmol) in of CH₂Cl₂ (5 ml) was cooled to 0° C. and treated with and DMDO in acetone (˜0.07 M, 15 eq., 4.5 mmol). The reaction was stirred for 2 days at RT. The mixture was concentrated to afford crude expoxide quantitatively. ¹H NMR (CD₃OD, 400 MHz): δ 1.11 (s, 3H), 1.16 (s, 3H), 1.54 (s, 3H), 2.07 (d, J=16.2, 1H), 2.25 (dd, J=16.2, J=1.6, 1H), 2.58 (d, J=19.1, 1H), 3.00 (d, J=19.1, 1H), 3.66 (d, J=1.6, 1H), 3.93 (d, J=8.5, 1H), 4.12 (s, 1H), 4.47 (d, J=8.5, 1H); ¹³C NMR (CD₃OD, 100 MHz): 16.1, 16.6, 17.9, 37.3, 38.6, 57.3, 64.8, 67.4, 69.4, 71.7, 75.8, 83.9, 108.3, 177.4, 180.2; IR (NaCl, cm⁻¹): 1772 s (C═O), 3410 br (O—H); MS Found: 295.0 (M+1), Calc. 294.11.

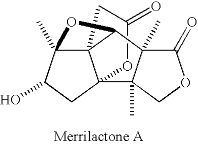

Merrilactone A (1). The crude epoxide 4 was stirred with p-TsOH.H₂O (80 mg, 0.42 mmol) in 25 mL of CH₂Cl₂ for 1 day at RT. The p-TsOH.H₂O was filtered off and washed 3 times with CH₂Cl₂. The crude product was chromatographed (CH₂Cl₂/AcOEt 4:1, then 2:1, then 1:1) to give merrilactone A (75 mg, 80% from alcohol 3).

Merrilactone A: ¹H NMR (CD₃OD, 400 MHz): δ 1.08 (s, 3H), 1.23 (s, 3H), 1.48 (s, 3H), 2.28 (dd, J=15.4, J=1.5, 1H), 2.68 (d, J=19.4, 1H), 2.70 (d, J=5.2, 1H), 2.73 (d, J=5.2, 1H), 2.90 (d, J=19.4, 1H), 3.94 (dd, J=5.2, J=1.5, 1H), 4.01 (d, J=10.1, 1H), 4.59 (d, J=10.1, 1H), 4.73 (s, 1H); ¹³C NMR (CD₃OD₁ 100 MHz): 16.1, 17.4, 17.5, 32.2, 43.9, 58.5, 61.2, 66.0, 75.4, 79.8, 90.1, 96.0, 107.1, 177.2, 178.9; IR (NaCl, cm⁻¹): 1766 s (C═O), 3448 br (O—H); MS Found: 295.19 (M+1), Calc. 294.11;

(R,R)-merrilactone A [α]D²¹=11.5 (c=0.17, MeOH)

(S,S)-Merrilactone A [α]D²¹=−11.8 (c=0.31, MeOH)

### REFERENCES

(1) Birman, V. B.; Danishefsky, S. J. J. Am. Chem. Soc. 2002, 124, 2080.
(2) (a) Hefti, F. Annu. Rev. Pharmacol. Toxicol. 1997, 37, 239. (b) Bennett, M. R.; Gibson, W. G.; Lemon, G. Auton. Neurosci. 2002, 95, 1. (c) Lu, P.; Blesch, A.; Tuszynski, M. H. J. Comp. Neurol. 2001, 436, 456. (d) Kaneko, M. J. Med. Chem. 1997, 40, 1863-9. (e) Backman, C.; Rose, G. M.; Hoffer, B. J.; Henry, M. A.; Bartus, R. T.; Friden, P.; Granholm, A. C. J. Neurosci. 1996, 16, 5437.
(3) (a) Huang, J.-M.; Yokoyama, R.; Yang, C.-S.; Fukuyama, Y. Tetrahedron Lett. 2000, 41, 6111. (b) Huang, J.-M.; Yang, C.-S.; Tanaka, M.; Fukuyama, Y. Tetrahedron 2001, 57, 4691.
(4) Murphy, Y. V. S. N.; Pillai, C. N. Synth. Commun. 1996, 26, 2363.
(5) (a) Denney, D. B.; Sherman, N. J. Org. Chem. 1965, 30, 3760. (b) Danishefsky, S. J.; Tsuzuki, K. J. Am. Chem. Soc. 1980, 102, 6893.
(6) Grieco, P. A.; Gilman, S.; Nishizawa, M. J. Org. Chem. 1976, 41, 1485.
(7) Wu, M. H.; Hansen, K. B.; Jacobsen, E. N. Angew. Chem. Int. Ed. 1999, 38, 2012.

1. An enantioenriched or enantiopure composition comprising a compound having the structure: 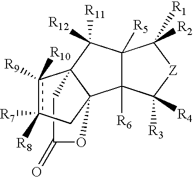
wherein Z is O or >N—X, where X is H, straight or branched substituted or unsubstituted alkyl, alkenyl or alkynyl, or acyl, carbamoyl, cycloalkyl, aryl, heterocycloalkyl, heteroaryl, aralkyl, amino, alkyl amino, or dialkyl amino;
wherein each of R₁ and R₂ is H or R₁ and R₂ together are ═O;
wherein each of R₃ and R₄ is H or R₃ and R₄ together are ═O;
wherein each of R₅ and R₆ are, independently, H, alkyl, aralkyl, or aryl;
wherein each of R₇ and R₈ is, independently, H, OH, or OR₁₄, where R₁₄ is alkyl or —C(O)—R₁₅,
where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino,
wherein each R₁₆ is straight or branched, substituted or unsubstituted alkyl, alkenyl or alkynyl, cycloalkyl, aryl, heterocycloalkyl, heteroaryl, aralkyl, or amino; and
wherein R₁₇ is straight or branched, unsubstituted alkyl, alkenyl or alkynyl, cycloalkyl, aryl, heterocycloalkyl, heteroaryl, aralkyl, or amino,
or wherein R₇ and R₉ together with the carbons to which each is attached form an oxirane moiety;
wherein each of R₉ and R₁₀ is, independently, H, alkyl, OH, or OR₁₃, where R₁₃ is an alkyl, an acyl, or an amide, or R₉ and R₁₀ together are ═CH₂,
or wherein R₈ and R₁₀ together with the carbons to which each is attached form an oxirane moiety;
wherein if one of R₇ or R₈ and one of R₉ or R₁₀ is absent, a double bond is formed as indicated by the broken line; and
wherein each of R₁₁ and R₁₂ is, independently, H, OH, or OR₁₃, where R₁₃ is an alkyl, an acyl, or an amide, or R₁₁ and R₁₂ together are ═O,
or wherein R₁₂ and R₁₀ together with the carbons to which each is attached form an oxetane moiety,
or an enantiomer, tautomer or salt of the compound,
wherein when the composition is enantiopure the composition is free of plant extracts.

2. The composition of claim 1, wherein in the compound when X is a substituted alkyl, substituents are selected from OH, oxo, halogen, alkoxy, diaklyamino or heterocyclyl.

3. The composition of claim 1, wherein in the compound Z is >N—X, where X is H, straight or branched substituted or unsubstituted alkyl, alkenyl or alkynyl, or acyl, carbamoyl, cycloalkyl, aryl, heterocycloalkyl, heteroaryl, aralkyl, amino, alkyl amino, or dialkyl amino.

4. The composition of claim 1, wherein in the compound Z is O or >N—X, where X is H, straight or branched alkyl, alkenyl or alkynyl, or acyl, carbamoyl, cycloalkyl, aryl, heterocycloalkyl, heteroaryl, aralkyl, amino, alkyl amino, or dialkyl amino;
wherein each of R₁ and R₂ is H or R₁ and R₂ together are ═O;
wherein each of R₃ and R₄ is H or R₃ and R₄ together are ═O;
wherein each of R₅ and R₆ are, independently, H, alkyl, or aralkyl;
wherein each of R₇ and R₈ is, independently, H, OH or OR₁₄,
where R₁₄ is alkyl or —C(O)—R₁₅,
where R₁₅ is H, —CH₂R₁₆, —CHR₁₆R₁₆, —CR₁₆R₁₇R₁₆, —OR₁₆, cycloalkyl, aryl, or aralkyl,
wherein each R₁₆ is alkyl, cycloalkyl, or aryl, aralkyl; and
wherein R₁₇ is alkyl, cycloalkyl, aryl, or aralkyl, or wherein R₇ and R₉ together with the carbons to which each is attached form an oxirane moiety;
wherein each of R₉ and R₁₀ is, independently, H, alkyl, OH, or OR₁₃, where R₁₃ is an alkyl, an acyl, or an amide, or R₉ and R₁₀ together are ═CH₂,
or wherein R₈ and R₁₀ together with the carbons to which each is attached form an oxirane moiety;
wherein if one of R₇ or R₈ and one of R₉ or R₁₀ is absent, a double bond is formed as indicated by the broken line; and
wherein each of R₁₁ and R₁₂ is, independently, H, OH, or OR₁₃, where R₁₃ is an alkyl, an acyl, or an amide, or R₁₁ and R₁₂ together are ═O,
or wherein R₁₂ and R₁₀ together with the carbons to which each is attached form an oxetane moiety.

5. The composition of claim 1, wherein the compound has the structure: 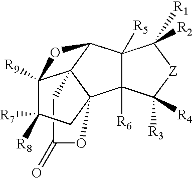
wherein Z is O;
wherein each of R₁ and R₂ is H, or R₁ and R₂ together are ═O;
wherein each of R₃ and R₄ is H, or R₃ and R₄ together are ═O;
wherein each of R₅ and R₆ are, independently, H, alkyl, aralkyl, or aryl;
wherein each of R₇ and R₈ is, independently, H, OH or OR₁₄,
where R₁₄ is alkyl or —C(O)—R₁₅,
where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino,
wherein each R₁₆ is straight or branched, substituted or unsubstituted alkyl, alkenyl or alkynyl, cycloalkyl, aryl, heterocycloalkyl, heteroaryl, aralkyl, or amino; and
wherein R₁₇ is straight or branched, unsubstituted alkyl, alkenyl or alkynyl, cycloalkyl, aryl, heterocycloalkyl, heteroaryl, aralkyl, or amino; and
wherein R₉ is H, alkyl, OH, or OR₁₃, where R₁₃ is an alkyl, an acyl, or an amide.

6. (canceled)

7. (canceled)

8. The composition of claim 1, wherein the composition is enantioenriched with an enantiomer having the structure:

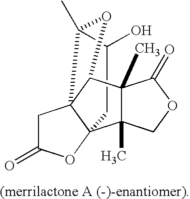

9. The composition of claim 1, wherein the composition is enantioenriched with an enantiomer having the structure:

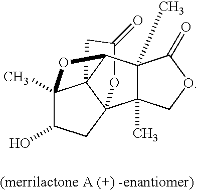

10-13. (canceled)

14. The composition of Claim 1 wherein the compound has the structure 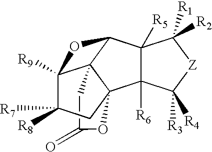
wherein Z is O;
wherein each of R₁ and R₂ is H, or R₁ and R₂ together are ═O;
wherein each of R₃ and R₄ is H, or R₃ and R₄ together are ═O;
wherein each of R₅ and R₆ are, independently, H, alkyl, aralkyl, or aryl;
wherein each of R₇ and R₈ is, independently, H, OH or OR₁₄,
where R₁₄ is alkyl or —C(O)—R₁₅,
where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino,
wherein each R₁₆ is straight or branched, substituted or unsubstituted alkyl, alkenyl or alkynyl, cycloalkyl, aryl, heterocycloalkyl, heteroaryl, aralkyl, or amino; and
wherein R₁₇ is straight or branched, unsubstituted alkyl, alkenyl or alkynyl, cycloalkyl, aryl, heterocycloalkyl, heteroaryl, aralkyl, or amino; and
wherein R₉ is H, alkyl, OH, or OR₁₃, where R₁₃ is an alkyl, an acyl, or an amide.

15. The composition of claim 1, wherein R₉ is H, alkyl or OR₁₃, where R₁₃ is an alkyl, an acyl, or an amide.

16. The composition of claim 1,
wherein R₁ and R₂ together are ═O;
wherein each of R₃ and R₄ is H;
wherein each of R₅ and R₆ are, independently, H, alkyl, or aralkyl;
wherein each of R₇ and R₈ is, independently, H, OH or OR₁₄,
where R₁₄ is alkyl or —C(O)—R₁₅,
where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino,
wherein each R₁₆ is straight or branched, substituted or unsubstituted alkyl, alkenyl or alkynyl, cycloalkyl, aryl, heterocycloalkyl, heteroaryl, aralkyl, or amino; and
wherein R₁₇ is straight or branched, unsubstituted alkyl, alkenyl or alkynyl, cycloalkyl, aryl, heterocycloalkyl, heteroaryl, aralkyl, or amino; and
wherein R₉ is alkyl.

17-24. (canceled)

25. A process for preparing a racemic composition comprising an equimolar mixture of a pair of enantiomers having the structures: 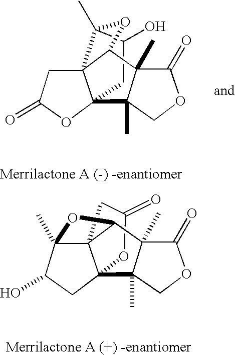
comprising:
a) reacting 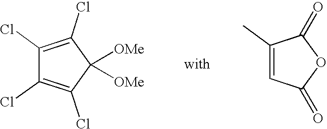
at a temperature of 140° C. to 230° C. to produce a compound having the structure: 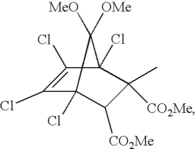
b) stereospecifically C-methylating the compound produced in step a) to produce a compound having the structure: 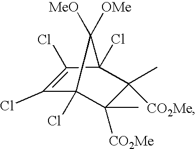
c) treating the compound produced in step b) with a suitable source of hydride and refluxing, and then with Na, NH₃ or Na, EtOH or L₁, NH₃ to produce a compound having the structure: 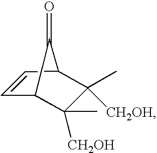
d) treating the compound produced in step c) with 2,2-dimethoxypropane, acetone and pTsOH, then treating the compound with NaH, (EtO)₂POCH₂CO₂Et, and THF, and then treating the compound with Mg and MeOH to produce a compound having the structure: 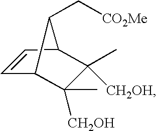
e) oxidizing the compound produced in step d) to produce a compound having the structure: 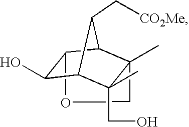
f) oxidizing the compound produced in step e) with PDC and DMF and then esterifying the product with K₂CO₃, MeI and acetone to give a compound having the structure: 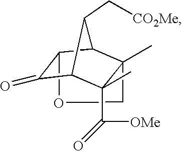
g) oxidizing the compound produced in step f) with magnesium monoperoxyphthalate hexahydrate and MeOH at −10° C. to +10° C. to produce a compound having the structure: 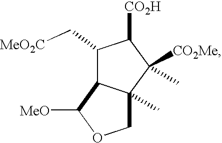
h) treating the compound produced in step g) with DCC and mCPBA at −10° C. to +10° C., and then treating the product with PhH, and then treating the product with K₂CO₃ and MeOH to produce a compound having the structure: 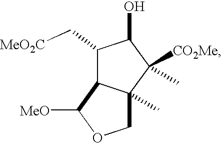
i) treating the compound produced in step h) with BF₃.OEt₂, or TiCl₄ or PTsOH to produce a compound having the structure: 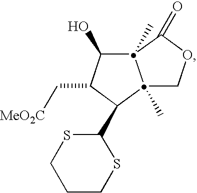
j) treating the compound produced in step i) with PhI(OCF₃CO₂)₂ and CH₃CN/H₂O, and then with NaBH₄ and MeOH at −10° C. to +10° C. to produce a compound having the structure: 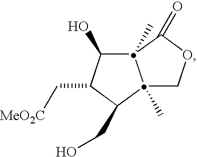
k) treating the compound produced in step j) with o-NO₂C₆H₄SeCN, Bu₃P, and THF, then 25%-35% H₂O₂, then treating the compound with a silyl protecting group, Et₃N and CH₂Cl₂ to produce a compound having the structure: 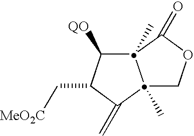
where Q is a silyl protecting group,
l) treating the product of step k) to LiOH, MeOH/H₂O and then I₂ to produce a compound having the structure: 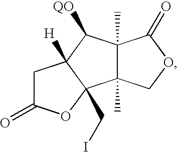
m) processing the product of step 1) to produce the racemic composition;
or
a process for preparing enantiopure merrilactone A or an enantioenriched composition of merrilactone A enantiomer comprising:
a) reacting 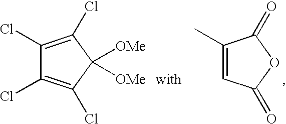
at a temperature of 140° C. to 230° C. to produce a compound having the structure: 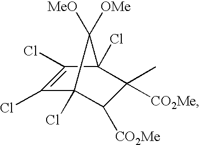
b) stereospecifically C-methylating the compound produced in step a) to produce a compound having the structure: 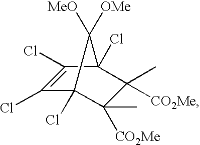
c) treating the compound produced in step b) with a suitable source of hydride and refluxing, and then with Na, NH₃ or Na, EtOH or L₁, NH₃ to produce a compound having the structure: 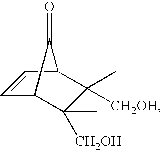
d) treating the compound produced in step c) with 2,2-dimethoxypropane, acetone and pTsOH, then treating the compound with NaH, (EtO)₂POCH₂CO₂Et, and THF, and then treating the compound with Mg and MeOH to produce a compound having the structure: 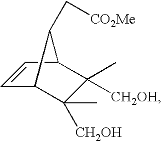
e) treating the compound produced in step d) with dimethyldioxirane and CH₂Cl₂ to give a compound having the structure: 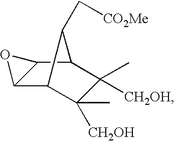
f) exposing the compound produced in step e) to either (S,S)-[Co^{III}(salen)]-OAc or (R,R)-[Co^{III}(salen)]-OAc at −110° C. to −55° C., and then to THF −45° C. to −5° C. to give an enantiomeric enriched compound having the structure: 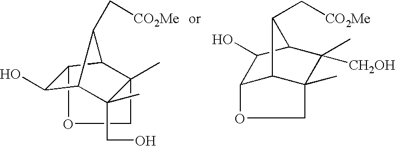
g) oxidizing the compound produced in step f) with PDC and DMF and then esterifying the product with K₂CO₃, MeI and acetone to give a compound having the structure: 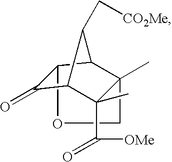
h) treating the compound produced in step g) with magnesium monoperoxyphthalate hexahydrate and MeOH at −10° C. to +10° C. to produce a compound having the structure: 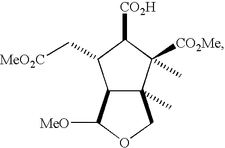
i) treating the compound produced in step h) with DCC and mCPBA at −10° C. to +10° C., and then refluxing the compound with PhH, and then treating the compound with K₂CO₃ and MeOH to produce a compound having the structure: 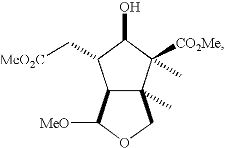
j) treating the compound produced in step i) with BF₃.OEt₂, or TiCl₄ or PTsOH to produce a compound having the structure: 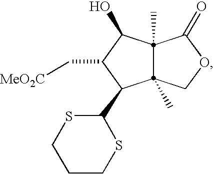
k) treating the compound produced in step j) with PhI(OCF₃CO₂) 2 and CH₃CN/H₂O, and then with NaBH₄ and MeOH at −10° C. to +10° C., to produce a compound having the structure: 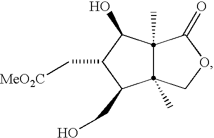
l) treating the compound produced in step k) with o-NO₂C₆H₄SeCN, Bu₃P, and THF, then 25%-35% H₂O₂, then treating the compound with a silyl protecting group, Et₃N and CH₂Cl₂ to produce a compound having the structure: 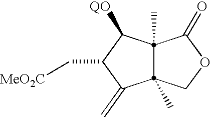
where Q is a silyl protecting group,
m) treating the product of step 1) with LiOH, MeOH/H₂O and then I₂ in saturated NaHCO₃/THF, a compound having the structure: 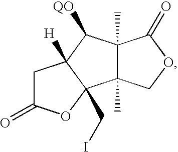
n) processing the product of step m) to produce the composition enantioenriched with a (+)-enantiomer or a (−)-enantiomer of the merrilactone A, and optionally purifying the (+)-enantiomer or a (−)-enantiomer of the merrilactone A to produce the enantiopure merrilactone A.

26. (canceled)

27. (canceled)

28. The process of claim 25, wherein step a) comprises treating in the presence of MeOH, then refluxing, then treating in the presence PhH-Me-OH then TMSCHN₂.

29. The process of claim 25, wherein the compound in step b) is stereospecifically C-methylated using LDA, HMPA, MeI, and THF at −110° C. to −55° C.

30. The process of claim 25, wherein the oxidizing in step d) is performed using mCPBA and CH₂Cl₂.

31. (canceled)

32. A compound having the structure: 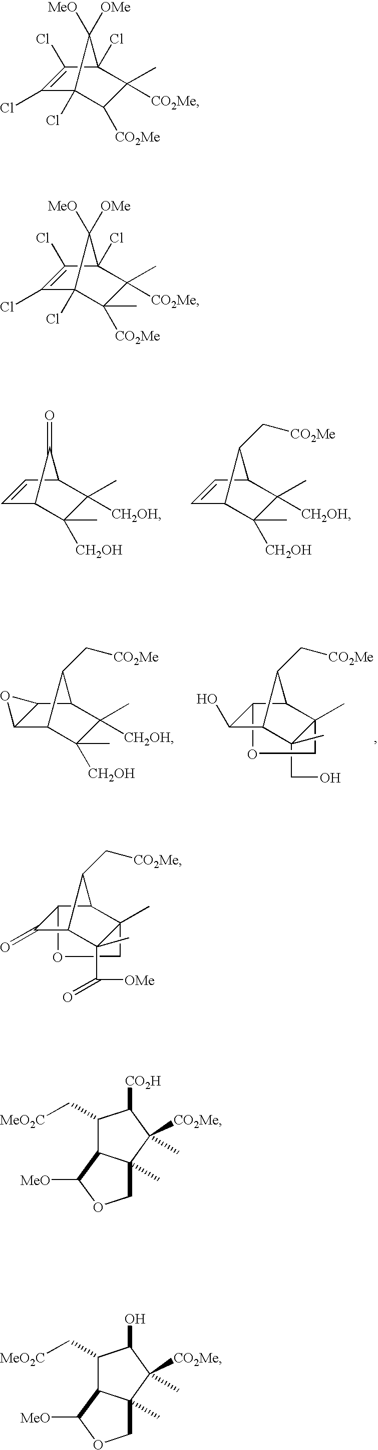 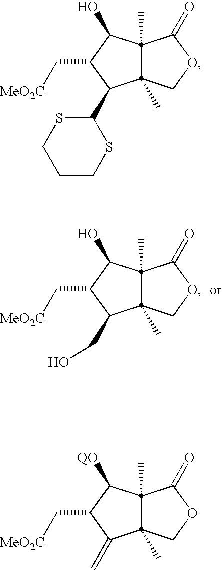
where Q is a silyl protecting group,
or an enantiomer thereof.

33. A method of alleviating a side effect resulting from a therapy-induced neuropathy in a patient receiving the therapy comprising administering to the patient the composition of claim 1 in an amount effective to alleviate the side effect.

34. The method of claim 33, wherein the therapy is a chemotherapy.

35-38. (canceled)

39. A method of treating a peripheral neuropathy in a patient suffering therefrom comprising administering to the patient the composition of claim 1 in an amount effective to treat the peripheral neuropathy.

40. A composition comprising an enantiopure compound free of plant extracts having the structure: 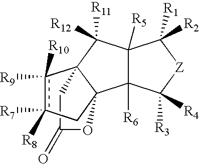
wherein Z is O or >N—X, where X is H, straight or branched substituted or unsubstituted alkyl, alkenyl or alkynyl, or acyl, carbamoyl, cycloalkyl, aryl, heterocycloalkyl, heteroaryl, aralkyl, amino, alkyl amino, or dialkyl amino;
wherein each of R₁ and R₂ is H or R₁ and R₂ together are ═O;
wherein each of R₃ and R₄ is H or R₃ and R₄ together are ═O;
wherein each of R₅ and R₆ are, independently, H, alkyl, aralkyl, or aryl;
wherein each of R₇ and R₈ is, independently, H, OH or OR₁₄, where R₁₄ is alkyl or —C(O)—R₁₅,
where R₁₅ is H, —CH₂R₁₆, —CHR₁₆R₁₆, —CR₁₆R₁₇R₁₆, —OR₁₆, alkenyl or alkynyl, cycloalkyl, aryl, heterocycloalkyl, heteroaryl, aralkyl, amino, alkyl amino, or dialkyl amino,
wherein each R₁₆ is straight or branched, substituted or unsubstituted alkyl, alkenyl or alkynyl, cycloalkyl, aryl, heterocycloalkyl, heteroaryl, aralkyl, or amino; and
wherein R₁₇ is straight or branched, unsubstituted alkyl, alkenyl or alkynyl, cycloalkyl, aryl, heterocycloalkyl, heteroaryl, aralkyl, or amino,
or wherein R₇ and R₉ together together with the carbons to which each is attached form an oxirane moiety;
wherein each of R₉ and R₁₀ is, independently, H, alkyl, OH, or OR₁₃, where R₁₃ is an alkyl, an acyl, or an amide, or R₉ and R₁₀ together are ═CH₂,
or wherein R₈ and R₁₀ together with the carbons to which each is attached form an oxirane moiety;
wherein if one of R₇ or R₈ and one of R₉ or R₁₀ is absent, a double bond is formed as indicated by the broken line; and
wherein each of R₁₁ and R₁₂ is, independently, H, OH, or OR₁₃, where R₁₃ is an alkyl, an acyl, or an amide, or R₁₁ and R₁₂ together are ═O,
or wherein R₁₂ and R₁₀ together with the carbons to which each is attached form an oxetane moiety, or an enantiomer, tautomer or salt of the compound.

41. A process for preparing an enantiopure merrilactone A or an enantioenriched composition of a merrilactone A enantiomer, comprising:
a) reacting 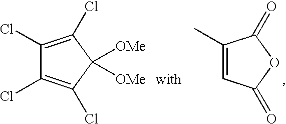
at a temperature of 140° C. to 230° C. to produce a compound having the structure: 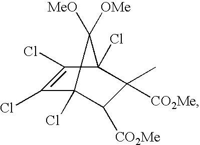
b) stereospecifically C-methylating the compound produced in step a) to produce a compound having the structure: 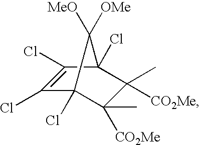
c) treating the compound produced in step b) with a suitable source of hydride and refluxing, and then with Na, NH₃ or Na, EtOH or L₁, NH₃ to produce a compound having the structure: 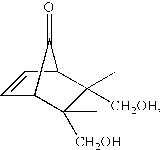
d) treating the compound produced in step c) with 2,2-dimethoxypropane, acetone and pTsOH, then treating the compound with NaH, (EtO)₂POCH₂CO₂Et, and THF, and then treating the compound with Mg and MeOH to produce a compound having the structure: 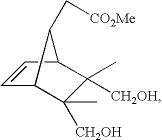
e) treating the compound produced in step d) with dimethyldioxirane and CH₂Cl₂ to give a compound having the structure: 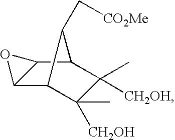
f) exposing the compound produced in step e) to either (S,S)-[CoIII(salen)]-OAc or (R,R)-[CoIII(salen)]-OAc at −110° C. to −55° C., and then to THF −45° C. to −5° C. to give an enantiomeric enriched compound having the structure: 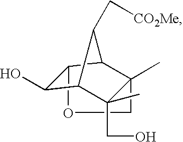
g) oxidising the compound produced in step f) with PDC and DMF and then esterifying the product with K₂CO₃, MeI and acetone to give a compound having the structure: 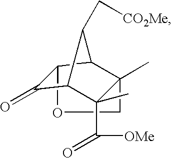
h) treating the compound produced in step g) with magnesium monoperoxyphthalate hexahydrate and MeOH at −10° C. to +10° C. to produce a compound having the structure: 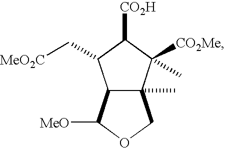
i) treating the compound produced in step h) with DCC and mCPBA at −10° C. to +10° C., and then refluxing the compound with PhH, and then treating the compound with K₂CO₃ and MeOH to produce a compound having the structure: 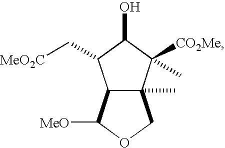
j) treating the compound produced in step i) with BF₃.OEt₂, or TiCl₄ or PTsOH to produce a compound having the structure: 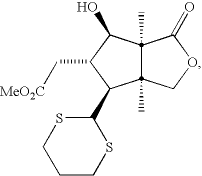
k) treating the compound produced in step j) with PhI(OCF₃CO₂)₂ and CH₃CN/H₂O, and then with NaBH₄ and MeOH at −10° C. to +10° C., to produce a compound having the structure: 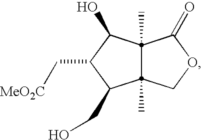
l) treating the compound produced in step k) with o-NO₂C₆H₄SeCN, Bu₃P, and THF, then 25%-35% H₂O₂, then treating the compound with a silyl protecting group, Et₃N and CH₂Cl₂ to produce a compound having the structure: 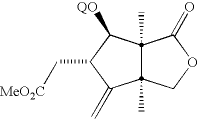
where Q is a silyl protecting group,
m) treating the product of step 1) with LiOH, MeOH/H₂O and then 12 in saturated NaHCO₃/THF, a compound having the structure: 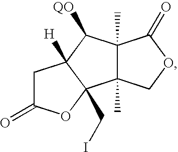
n) processing the product of step m) to produce the composition enantioenriched with a (+)-enantiomer or a (−)-enantiomer of the merrilactone A, and optionally purifying the (+)-enantiomer or a (−)-enantiomer of the merrilactone A to produce the enantiopure merrilactone A.
